(19) United States
(12) Patent Application Publication
(10) Pub. No.: US 2006/0115814 A1
(43) Pub. Date: Jun. 1, 2006

(54) Methods and compositions, relating to hiv gp41 antigens and other hiv envelope antigens

(76) Inventors: Miles W. Cloyd, Alvin, TX (US); Jianmin Chen, Galveston, TX (US)

Correspondence Address: FULBRIGHT & JAWORSKI L.L.P., 600 CONGRESS AVE., SUITE 2400, AUSTIN, TX, 78701, US

(21) Appl. No.: 10/506,095

(22) PCT Filed: Feb. 26, 2003
(86) PCT No.: PCT/US03/06206

§ 371 Date: Nov. 21, 2005

Related U.S. Application Data
(60) Provisional application No. 60/360,448, filed on Feb. 28, 2002.
(60) Provisional application No. 60/373,448, filed on Apr. 18, 2002.

(51) Int. Cl. C12Q 1/70; C12Q 1/68

(52) U.S. Cl. 435/6; 435/5

(57) ABSTRACT

The present invention concerns methods and compositions involving nondenatured IIIV gp41 antigens alone and in combination with other nondenatured HIV envelope antigens for the detection of early antibodies against HIV. Such methods and compositions may be used to detect HIV infection in a patient or in a blood sample. The compositions of the invention allow for the detection of antibodies at a stage at which they were previously undetectable. The present invention also concerns kits for implementing such methods. In some embodiments, kits contain a recombinant, nondenatured gp41 antigen and a recombinant, denatured gp160 antigen.

The present application claims the benefit of priority to U.S. Provisional Patent Applications 60/360,448 filed on Feb. 28, 2002 and 60/373,448 filed Apr. 18, 2002 which are specifically incorporated by reference herein.

### BACKGROUND OF THE INVENTION

1. Field of the Invention

The present invention relates generally to the fields of virology and immunology. More particularly, it concerns a gp41 antibody in compositions and methods for screening patients for HIV.

2. Description of Related Art

Humans infected with the Human Immunodeficiency Virus (HIV) generally mount a humoral immune response to the virus, resulting in production of specific antibodies. Since the presence of antibody to HIV-1 is a marker for virus infection, the FDA has approved anti-HIV antibody testing as a method of screening donated blood for the virus (Schleupner 1989; Steckelberg et al., 1988). Consequently, enzyme-linked immunoabsorbent assays (EIA) have been used as the principle diagnostic tool by clinicians to detect HIV-1 infection. The frequency of false positive results of the early EIAs resulted in the requirement that confirmatory testing, either by Western blot (WB) or fixed-cell immunofluorescence, be performed for diagnosis. While the newer EIAs are more specific (Litvak et al., 1997), they fail to detect antibody in individuals who are very early in the course of their infection, during a “window” of time between infection with HIV-1 and the production of serum antibodies detectable by current commercial EIA/WB (seroconversion), or who are infected with certain HIV-1 clades (Aiuti et al., 1993; Kura et al., 1998; Rich et al., 1998; Urnovitz et al., 1997; Yerly et al., 1999; Laperche et al., 1998). The lack of early detectability remains a persistent public health issue among recipients of blood or organ donations, and raises concerns among health care workers who have been envelope antigen of an HIV-infected cell, such that it has less than 50%, 40%, 30%, 20%, 10%, 5%, or 1% of the binding activity with a particular HIV antibody, such as one present three weeks after infection, as compared to the native form. The particular infected cell may be selected from a cell that has been infected with an HIV including, but not limited to, the following subtypes: HIV_{MCK}, HIV_{PM16}, HIV_{PM205}, HIV₂₁₃, HIV_{ED-1}, HIV_{TP-1}, HIV_{AK-1}, HIV_{SK-1}, HIV_{AC-1}, HIV₂₁₄, HIV_{O}, HIV_{G1}, or HIV_{C}. The native form of an envelope antigen of an HIV-infected cell are envelope antigens that have not been treated with a reducing buffer, ionic buffer or high levels of ionic or non-ionic detergents (e.g., greater than 5% NP-40 or 10% digitonin or 2.0% deoxycholate) or any buffer or agent that would destroy the configurational integrity (i.e., quaternary, tertiary or secondary structure) of the native antigen peptide. The denatured HIV antigen is further described as a protein (p) or glycoprotein (gp) having a particular molecular weight in daltons of its given numerical designation multiplied by 1,000. Many of these denatured HIV antigens are used in monitoring HIV infection. Some of these include p17, p19, p24, p38, gp41 and p55. Applicants have described the invention as detecting anti-HIV antibodies in serum samples seronegative for antibodies to these denatured HIV antigens. Denatured HIV antigens are further described as native HIV antigens that have been extracted with such reducing agents as dithiothreitol or ionic detergent, or buffers containing high concentrations of non-ionic detergent (e.g., greater than 5% NP-40 or 10% digitonin or 2.0% deoxycholate) or any other buffer or agent that would destroy the conformational integrity (e.g., quaternary, tertiary or secondary structure) of the native antigen peptide.

The “native” form of the HIV-infected cell envelope antigen is maintained through use of a phosphate buffer containing low levels of a non-ionic detergent (e.g., less than about 5% NP-40, 10% digitonin, or 2.0% deoxycholate). Ionic detergents are employed to selectively degrade immunoreactive epitope(s). The loss of immunoreactivity after each treatment can be measured in parallel with “native” envelope protein. However, even mild denaturing treatments may destroy the antigenic epitopes recognized by “early” HIV immune sera, especially where confornational patterns involving non-contiguous sequences within the protein are important.

The nondenatured HIV envelope antigens, such as gp41 or the combination of gp41 and gp160, of the invention may be capable of specifically binding an “early HIV antibody.” See Race et al., 1991. They may be capable of specifically binding an HIV antibody within 1, 2, 3, 4, 5, 6, 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more weeks of infection. The nondenatured antigens may be capable of specifically binding or recognizing an HIV antibody as early as within 1-50 weeks of infection, within 5-30 weeks of infection, or within 10-20 weeks of infection, though such antigens may also be capable of binding HIV antibodies at later times. The nondenatured HIV envelope antigens of the invention may also be capable of specifically binding an HIV antibody 1, 2, 3, 4, 5, 6, 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more weeks earlier (based on when samples were collected from the same individual, as described in Example 6) than a denatured antigen, for example, one prepared using greater than 2% SDS and employed in a standard EIA assay. It is contemplated that any BBI panel having samples obtained at different time points may be used in a standard EIA assay to evaluate when a nondenatured antigen of the invention binds an HIV antibody as compared to a denatured antigen, such as those used in commercially available assays. A 2002 BBI catalog is included as Appendix A, and is specifically incorporated by reference. Nondenatured antigens are also capable of detecting HIV infection in a sample earlier than commercially available tests, including denatured HIV antigen, antibody, and PCR RNA tests. The invention covers nondenatured antigens that retain their conformation to bind specifically an HIV antibody previously undetectable or undetectable at a particular time of infection compared to other antigens. Thus, the present invention concerns screening methods in which the recombinant, nondenatured HIV envelope antigen is capable of specifically binding an antibody from a sample from a subject collected 2 to 50 days earlier than a sample from the same subject in which a denatured gp41 or gp160 antigen is capable of specifically binding an antibody. Alternatively, methods of the invention concern samples from individuals infected with HIV for years, but also infected for fewer than 16 weeks when the samples were collected.

Thus, in some embodiments of the invention, compositions include a nondenatured gp41 antigen. It is contemplated that the gp41 antigen may comprise all or part of a gp41 peptide or polypeptide sequence. In some embodiments, the gp41 antigen comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, or 135 contiguous amino acids of SEQ ID NO:1 (edline entry AAA76690 comprising all of gp160, which is specifically incorporated by reference), SEQ ID NO:2 (amino acids 511-856 inclusive of Medline entry AAA76690, thus SEQ ID NO:2 is subsumed by SEQ ID NO:1), or any of SEQ ID NOS:3-12. In some embodiments, the gp41 antigen comprises the amino acid sequence of SEQ ID NO:2. The entire amino acid sequence of a gp41 polypeptide corresponds to amino acids 511 to 856, inclusive, of SEQ ID NO:1; this sequence is also referred to as SEQ ID NO:2. Thus, in further embodiments, a gp41 antigen comprises all or part of SEQ ID NO:2 or all or part of the 511-856 amino acid region (inclusive) of SEQ ID NO:1.

It is contemplated that any embodiments discussed or described in the context of one SEQ ID NO, may be implement or applied to with respect to any other SEQ ID NO disclosed herein, to the extent possible.

In further embodiments of the invention, compositions include a nondenatured gp160 antigen. It is contemplated that the gp160 antigen may comprise all or part of a gp41 peptide or polypeptide and/or a gp120 peptide or polypeptide sequence. In some embodiments, the gp160 antigen comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 375, 400, 425, 450, 475, 500, 525, 550, 575, 600, 625, 650, 675, 700, 725, 750, 775, 800, 825, 850, or 856 contiguous amino acids of SEQ ID NO:1-12. In some embodiments, the gp160 antigen comprises the amino acid sequence of any of SEQ ID NO:2-12.

Compositions of the invention include, in some embodiments, multiple HIV envelope protein (gp 41 and/or gp160) antigens, such as 1, 2, 3, 4, 5, 6, 7, 8, 9, 10, 11, 12, 13, 14, 15, 16, 17, 18, 19, 20 or more antigens in the compositions, which may be from a single HIV envelope polypeptides or from different HIV envelope polypeptides. It is further contemplated that antigens may be from the same type of HIV envelope polypeptide but from different clades. For example, an antigen from gp41 of HIV-1 group O may be included in the same composition comprising an gp41 antigen (homologous or different region) of HIV-1 group B. Alternatively, the composition may comprise one or more antigens from gp41 and one or more antigens from gp160 (from the same or different subtype). It is contemplated that antigens from multiple subtypes may be contained in a single composition. It is further contemplated that antigens may not be derived from any single subtype but may be variations of subtype, for example, by doing a sequence comparison of a particular antigen domain between different subtypes and designing a sequence based on that comparison. Thus, an antigen sequence may be optimized based on the amino acids with the greatest identity at particular positions. The invention is premised on the concept that any antigen that is recognized by an HIV antibody directed to any nondenatured HIV envelope protein may be utilized in the invention, whether wild-type or not.

Antigens from any and all clades or strains (refers synonymously to “subtypes”) are contemplated as part of the invention as different sequences should be operable with respect to the invention, though some may provide more sensitive assays than others. Subtypes include, but are not limited to: HIV_{MCK}, HIV_{PM16}, HIV_{PM205}, HIV₂₁₃, HIV_{ED-1}, HIV_{TP-1}, HIV_{AK-1}, HIV_{SK-1}, HIV_{AC-1}, HIV₂₁₄, HIV_{O}, HIV_{G1}, or HIV_{C}.

Antigens of the invention are recombinant in some embodiments of the invention. The term “recombinant” refers to nucleic acid or proteinaceous compound that has been manipulated in vitro. If a proteinaceous composition is expressed from a nucleic acid sequence that has been recombinantly manipulated, the proteinaceous composition is recombinant. In some embodiments of the invention, a recombinant, nondenatured lysates of such transfected eukaryotic cells. As used in the description of the present invention in the description of the human immunodeficiency virus recombinant protein/peptide preparations and cell lysates, the phrase “substantially nondenatured” in used to define a peptide or protein having a preserved configurational integrity of the human immunodeficiency virus envelope gp160 protein or a portion thereof sufficient to bind early anti-HIV antibody. It is specifically contemplated that the “substantially nondenatured” HIV envelope antigens does not include HIV envelope antigens disclosed in the prior art that are capable of binding an HIV antibody, but at a diminished capacity than those prepared under nondenaturing conditions. Nondenaturing conditions enhance the detection of immunoreactivity between the HIV envelope antigens of the invention and HIV antibodies, while providing for formation of secondary structure related to an involved epitope. Conformation of the protein/peptide used as target antigen is important in providing this early antibody recognition.

The present invention provides procedures that preserve sufficient conformational integrity of the protein/peptide to allow early anti-HIV binding recognition. It is anticipated that given the disclosure here, other similar protein/peptide preparation processes may be devised that result in useful target antigen compositions for early anti-human immunodeficiency virus screening. All such modified procedures, insofar as they represent minor or insignificant modification of the procedures and specific materials described herein, are therefore intended by the inventor to be embraced within the scope of the present invention.

While a number of different HIV strains were examined by the present inventor, other HIV viral strains not specifically mentioned or examined here may also be employed in the preparation of the various HIV proteins/peptides of the invention. It is expected that other HIV viral strains may be used to provide the defined substantially preserved conformational epitopically intact HIV proteins capable of “early anti-HIV antibody” (“early anti-HIV antibody” and early HIV antibody” are synonymous) recognition. The particularly noted HIV strains used to create recombinant protein/peptide target antigen were selected based on an observed activity to bind “early anti-HIV antibody” in human patient serum or plasma samples determined to be seronegative by conventional antibody testing procedures. Hence, additional such representative strains may be identified and selected using the procedures outlined herein, and subsequently processed again according to the procedures described in detail here in providing recombinant HIV antigen also useful in screening and diagnosing early anti-HIV antibody and HIV infection in a patient sample.

Methods of the invention include screening a sample for HIV antibodies using any of the compositions described above.

In some methods of the invention, a sample is contacted with a composition comprising a recombinant, nondenatured HIV envelope protein antigen under conditions that permit formation of an immunocomplex between any antibody in the sample that can specifically bind to the antigen. The method further involves detecting whether an immunocomplex is formed between an antibody and the antigen. In some embodiments, the sample may be contacted with a second, third, fourth, fifth, or more HIV envelope antigen, separately or in the same composition as the the first antigen. The sample may be any sample suspected of containing HIV antibodies, including blood, serum, saliva, tears, semen, cervical fluid, vaginal swab or lavage, or placenta. The sample may be from a subject, including humans. The subject may also be an infant or a subject afflicted with idiopathic chronic lymphopenia or suspected of being afflicted with that condition.

The step of determining whether an immunocomplex is formed may be accomplished by a number of ways well known to those of ordinary skill in the art. In some embodiments, the immunocomplex is detected using ELISA or Western blotting. In other embodiments, it is accomplished using an anti-antibody second reagent, which refers to a compound tha specifically binds an antibody. Compounds of the invention may be labelled with a detecting agent, which may be calorimetric, enzymatic, radioactive, chromatographic, or fluorescent. The antigen may be affixed to a solid, nonreactive support, which refers to a compound that will not react with antigens of the invention or antibodies in any sample. The support may be a plate or assay dish, and may be made of any nonreactice material, including, glass, plastic, and silicon.

Embodiments of the invention also include kits comprising any of the components of the invention described above, in a suitable container means. Kits may include one or more HIV antigens. In some embodiments, antigens are from the same polypeptide, such as gp41, while in other embodiments, antigens are from both gp41 and gp160 or gp120. In still further embodiments, antigens are from the same or different strains. Such antigens may be in the same or in separate compositions. Kits may further include non-reactive supports in which antigens of the invention are affixed or attached. Kits may also include secondary antibody reagents. Antigens or antibodies in the kits may be labelled. Labels may be colorimetric, enzymatic, radioactive, or fluorescent.

It is contemplated that any feature discussed with respect to one embodiment of the invention may be employed with any other embodiment of the invention described herein. Furthermore, compositions and methods of the invention may be employed interchangeably.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Detection of ““early HIV antibodies”” in sera of some high-risk subjects by immunofluorescence staining of live HIV-infected H9 cells or HIV env-expressing CEM cells. Live H9 T cells infected with HIV_{213, AC-1}, and _{C} at the peak of virus production were used as targets and stained with normal human serum (NHS), sera from WB-negative high-risk subjects (R299, R343, and R359), and a serum from an HIV WB-positive patient (R399) (all at 1:30 dilutions). HIV env-expressing cell lines³⁸ (CEM-213 env+CEM-AC-1 env) were used as targets in live-cell IFA. (Filled histogram=uninfected control cells. Open histogram=HIV-infected or env-expressing cells).

FIG. 2. Evaluation of preservation of HIV epitopes reacting with ““early HIV antibodies””. HIV-₂₁₃-and HIV_{AC-1}-infected H9 cells were labeled with ³⁵S- methionine and lysed in the indicated detergents, followed by radioimmunoprecipitation (RIP) with subject R6 serum (containing ““early HIV antibodies””) and control normal serum (NS). The precipitates were analyzed by SDS/PAGE.

FIG. 3. inmunoprecipitation of HIV-infected Cells Solublized in Different Detergents analyzed by SDS-PAGE with a Western Blot Read-out. CEM cells uninfected or infected with HIV strains 213, AC-1 and C were lysed in various detergents and incubated overnight into either normal human serum (NS), “early HIV Ab”-positive serum (R299), or Western blot-positive serum (R310). The immunoprecipitation were captured by Pansorbin, washed, and analyzed by SDS-PAGE. The proteins were then transferred to a nitrocellulose filter by Western blotting and reacted to Mab against gp41 and gp120.

### DESCRIPTION OF ILLUSTRATIVE EMBODIMENTS

The present invention is based on the characterization of a nondenatured gp41 antigen from HIV, which carries ramifications particularly with respect to the diagnosis and treatment of HIV.

I. HIV

The present invention concerns compositions and methods for detecting human immunodeficiency virus (HIV) antibodies in subjects infected with the virus. It concerns particularly the use of nondenatured envelope protein antigens that can bind to antibodies in a subject infected with HIV for use in diagnostic and screening methods of the invention.

The general biology of the HIV retrovirus involves a genomic RNA molecule and various associated proteins that are encapsulated in a capsid of protein nature (nucleocapsid). The entire structure is protected by a membrane of cellular origin, which has incorporated the envelope protein of viral origin. Under physiologic conditions, the envelope protein (env) is initially synthesized in the form of a precursor, containing at its N-terminal end a signal sequence, which initiates the passage of the precursor into the endoplasmic reticulum (secretion route). This signal peptide is then removed by proteolytic cleavage. The product of this cleavage is a protein referred to as gp160, which is itself subsequently cleaved into a gp41 small subunit (also referred to as gp40 subunit) and a gp120 large subunit. The N-terminal end of the gp120 corresponds to the N-terminal end of the gp160, while the C-terminal end of the gp41 corresponds to the C-terminal end of the gp160.

There is a large amount of early data, as well as the more definitive needle-stick and blood transfusion data, which shows that there is an interval of time (termed the immunologically “silent window”) following HIV infection in which individuals do not score positive in serological tests employing denatured antigens (EIA, WB). This “silent” period has been reported to be from one to many months (Aiuti et al., 1993; Busch et al., 1995; Imagawa et al., 1989; Ensoli et al., 1990; Gorrino et al., 1994; Horsburgh et al., 1989; Loche et al., 1988; Mariotti et al., 1990; Mayer et al., 1986; Pezzella et al., 1991; Ranki et al., 1987; Salahuddin 1984; Wolinsky et al., 1989). There have been numerous reports of transmission of HIV from such individuals, either through blood transfusion, sexual activity, or organ donation (Aiuti et al., 1993; Busch et al., 1991; Sehgal, 1998; Barbiano di Belgiojoso et al., 1998; Cohen et al., 1989; Ward, 1993; Ward et al., 1988; Ensoli et al., 1991), and even now, with employment of 3^{rd} generation EIAs, case reports still appear (Ling et al., 2000; Murphy et al., 1998). A number of studies have attempted to shorten the “silent period” using PCR (Brettler et al., 1992; Eble et al., 1992; Gupta et al., 1992; Luque et al., 1993; Pan et al., 1991; Sheppard et al., 1993; Yerly et al., 1991; Coutlee et al., 1994), but, in general, routine, single amplification regimen PCR for HIV DNA does not detect infection much earlier than the newest EIAs (Coutlee et al., 1992). The new RT-PCR assays, however, for HIV RNA in plasma appear to shorten the “window” somewhat.

Recently, several 4^{th} generation EIAs (Saville et al., 2001; Weber et al., 1998; Binsbergen et al., 1999; Binsbergen et al., 1998; Gurtler et al., 1998; Brust et al., 2000) have been developed to detect HIV Ag and Ab simultaneously. One such test, VIDAS HIV DUO Ultra, uses native gp160 as one of the coating Ags and it can detect infection in 10 BBI seroconversion panels on average about 12 days earlier than the current antibody assays. However, these 10 seroconverter panels all possessed detectable HIV Ag at much earlier time points than Ab scored by current denatured Ag EIAs and this appeared to be a selected group of panels. Many studies have compared Ag detection assays and the denatured Ag EIAs for Abs, and found that sometimes Ag can be detected earlier than Ab and sometimes not. Two panels were detected earlier by the new 4^{th} generation test in comparison to the Ag test. An earlier version of this test, VIDAS HIV DUO which contains the same antigens as DUO ultra except no native gp160, could detect infection on average 4 days earlier than the 3^{rd} generation antibody assay in 5 of 12 BBI seroconversion panels tested. These results strongly suggest that it is primarily the antibody to the native gp160 component within the DUO Ultra 4^{th} generation format which provides the earliest detection of HIV infection, rather than the antigen assay component. Of particular interest is that within this antibody assay, full-length native gp160 was added as part of the coating antigens in addition to gp41 and gp36 polypeptides, which are also used in 3^{rd} generation antibody assays. It seems likely that it is the native gp160 added in the antibody assay component which provides the ability to detect HIV infection earlier than the 3^{rd} generation antibody assays. Our native gp160 assay showed further improvement over the VIDAS HIV DUO ULTRA may due to the increased sensitivity of a pure gp160 format.

Using the BBI seroconversion panels, we showed that native gp160 assays can detect HIV infection about 2-4 weeks earlier than both the HIV RNA and antigen assays as well as 4-6 weeks earlier than the antibody assays. Clearly the current HIV tests, even in the 4^{th} generation combined Ag/Ab format, needs to be amended to include native gp160s as Ab-detecting antigens to detect earliest HIV infection, and this should probably include gp160 from several HIV strains. This will allow treatment of HIV infection at the earliest stage possible.

Several other markers of an immune response to HIV also appear to be good indicators of early infection. In addition to our study showing the existence of “early anti-HIV antibodies,” T cells reactive to HIV peptides have been shown to be present in high-risk, EIA-negative individuals (Clerici et al., 1991; Clerici et al., 1994). Also, in some early-infected EIA-negative patients, B cells which make anti-HIV antibodies can be expanded from peripheral blood in vitro (Jehuda-Coehn et al., 1990). Antibodies to HIV Nef and p17 proteins have also been reported to be present before sero-conversion in EIA (Stramer et al., 1989; Ameisen et al., 1989). Consequently, several studies indicate that immune responses occur early after infection and these do not necessarily involve induction of antibodies that react to linear epitopes of HIV proteins.

A. Proteinaceous Compositions

In certain embodiments, the present invention concerns novel compositions comprising at least one proteinaceous molecule, such as a gp41 antigen alone or in combination with other HIV envelope proteins. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein. The term “antigen” refers to any substance or material that is specifically recognized by an antibody or T cell receptor. The term “epitope” or “antigenic determinant,” refers to a particular region or recognition site on the surface of an antigen to which the antibody or T-cell receptor binds. Thus, it is contemplated that the antigens of the invention may be truncations or only portions of a full-length polypeptide. For example, a “gp41 antigen” refers to a peptide or polypeptide containing contiguous amino acids of gp41, including at least one gp41 epitope, but it may be fewer than than a full-length amino acid sequence. Thus, a gp41 antigen may include a region of contiguous amino acids of any of SEQ ID NO:1-12. Similarly, a “gp160 antigen” refers to a peptide or polypeptide containing contiguous amino acids of gp160, including at least one gp 160 epitope, but it may be fewer than than the full-length amino acid sequence. A gp160 antigen may include a region of contiguous amino acids of SEQ ID NO:1.

SEQ ID NO:1 corresponds to protein accession number AAA76690, which is the sequence for the envelope glycoprotein of a 1987 HIV type 1 isolate. SEQ ID NO:2 corresponds to amino acids 511-856 of SEQ ID NO:1, which is a full-length gp41 polypeptide sequence. Immunogenic regions of HIV envelope proteins have been described, and the present invention includes antigens that include one or more such regions. Amino acids 588-606 of SEQ ID NO:1 encodes an-immunodominant region of gp41 identified from HIV-1 subtype B (SEQ ID NO:3). A two amino acid change of SEQ ID NO:3 allows HIV-1 subtype O antibodies to be recognized (SEQ ID NO:4). Another immunodominant region of gp41 that allows subtype O to be recognized is SEQ ID NO:5. A slightly different immunodominant region of a gp41 is SEQ ID NO:5, which allows subtype O to be recognized, and SEQ ID NO:6, which allows subtype B to be recognized. Other immunodominant regions include a region from amino acid 578 to 613 (SEQ ID NO:7) (Chang et al., 1985), amino acids 599-609 (SEQ ID NO:8) (Banapour et al., 1987), amino acids 583-603 (SEQ ID NO:9) (Wang et al., 1986), amino acid 598-609 (SEQ ID NO:10) (Gnann et al., 1987), and 604-618 (SEQ ID NO:11) (Narvanen et al., 1988) of SEQ ID NO:1. A synthesis of the data regarding this immunodominant regions suggests an overlap corresponding to amino acids 579-613 (SEQ ID NO:12).

In certain embodiments, a proteinaceous molecule comprising an HIV envelope antigen may comprise, be at least, or be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater contiguous amino acid residues, and any range derivable therein of SEQ ID NO:1, SEQ ID NO:2, or SEQ ID NO:3-12.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Encompassed by certain embodiments of the present invention are peptides, such as, for example, a peptide comprising all or part of an HIV envelope antigen (including at least one epitope) of any subtype or lade. Peptides of the invention may comprise, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 contiguous amino acids, including all or part of SEQ ID NO:1, SEQ ID NO:2, or SEQ ID NO:3-12.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β3-alanine, β3-Amino-propionic | AHyl | allo-Hydroxylysine |
| | acid | | |
| Abu | 2-Aminobutyric acid | 3Hyp | 3-Hydroxyproline |
| 4Abu | 4-Aminobutyric acid, piperidinic | 4Hyp | 4-Hydroxyproline |
| | acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosnie |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide. In still further embodiments, a proteinaceous compound may be purified to allow it to retain its native or nondenatured conformation. Such compounds may be recombinantly derived or they may be purified from endogenous sources.

In certain embodiments, the proteinaceous composition may comprise at least one antigen of gp41, gp120, or gp160 that is recognized by an antibody. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is also used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that it will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

1. Functional Aspects

When the present application refers to the function or activity of a substantially nondenatured HIV envelope antigen, it is meant that the molecule in question has the ability to bind specifically to an early HIV antibody, in addition to being able to bind non-early HIV antibodies (such as those recognized by denatured HIV envelope antigens). An “early HIV antibody” refers to an antibody induced by HIV infection and produced shortly thereafter that recognizes conformational epitopes of gp41 and gp160. It is distinguishable from other HIV antibodies, which may be recognized by native or denatured HIV envelope antigens, or both. For an early HIV antibody to bind to gp41 or gp160, at least part or all of the native folded structures of these molecules must be maintained. Alternatively, an “early HIV antibody” is defined as an antibody that is undetectable, when assayed using standard EIA methods (such as those disclosed in the Example section) and when compared to nondenatured antigens of the invention (which bind to early HIV antibodies), by a fully denatured HIV envelope antigen or by any non-native HIV envelope antigen commercially available at the time this application was filed. Commercially available antigens include, but are not limited to, those found in Abbott Laboratories' HIVAB HIV-1/HIV-2 (rDNA) EIA. It is further contemplated that nondenatured antigens of the invention are those that will specifically recognize and bind an early HIV antibody that is purified or prepared recombinantly.

The assays disclosed in the Examples provide non-limiting standards for identifying an “early HIV antibody,” which is also detectable by live cell immunofluorescence (direct or indirect) or radioimmunopreciptation performed under nondenaturing conditions.

2. Variants of HIV Envelope Antigens

Amino acid sequence variants of the polypeptides of the present invention can be substitutional, insertional or deletion variants. Deletion variants lack one or more residues of the native protein that are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alatine to serine; arginine to lysine; asparagine to glutamine or histidine; aspartate to glutamate; cysteine to serine; glutamine to asparagine or histidine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “functionally equivalent codon” is used herein to refer to codons that encode the same amino acid, such as the six codons for arginine or serine, and also refers to codons that encode biologically equivalent amino acids (see Table 2, below).

TABLE 2
Codon Table

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAG GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UGA UCC UCG UCU |
| Threonine | Thr | T | ACA ACC ACG ACU |
| Valine | Val | V | GUA GUG GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity, as discussed below. Table 2 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See e.g., Johnson (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the conformational properties of HIV envelope antigens, but with altered and even improved characteristics.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a region to facilitate purification of the fusion protein. Inclusion of a cleavage site at or near the fusion junction will facilitate removal of the extraneous polypeptide after purification. Other useful fusions include lining of functional domains, such as active sites from enzymes such as a hydrolase, glycosylation domains, cellular targeting signals or transmembrane regions.

4. Protein Purification

It is desirable to purify HIV envelope antigens or variants thereof. These techniques involve, at one level, the crude fractionation of the cellular milieu to polypeptide and non-polypeptide fractions. The invention is directed at preserving the conformation of HIV envelope antigens as much as possible so that they are substantially nondenatured.

Antigens of the invention may be purified using gentle, nondenaturing detergents, which include, but are not limited to, NP40 and digitonin. Infected or transfected host cells may be solubilized using a gentle detergent. The following conditions are considered “substantially denaturing” or “denaturing”: 10 mM CHAPS, 0.5% SDS, >2% deoxycholate, or 2.0% octylglucoside. Antigens prepared under such conditions would not be considered “nondenatured antigens.” Preparations of substantially nondenatured antigens of the invention may be accomplished using techniques described in U.S. Pat. Nos. 6,074,646 and 5,587,285, which are hereby incorporated by reference herein.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

5. Antibodies

The present invention concerns the detections of HIV antibodies (anti-HIV antibodies) using substantially nondenatured HIV antigens, particularly those that were previously undetectable. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. As described earlier, an antigen may include one or more epitopes and an antigen refers to any part of a polypeptide that contains at least one epitope.

The term “antibody” is used to refer to any antibody-like molecule that has an antigen binding region. The techniques for preparing and using various antibody-based constructs and fragments are well known in the art. Means for preparing and characterizing antibodies are also well known in the art (See, e.g., Harlow and Lane, “Antibodies: A Laboratory Manual,” Cold Spring Harbor Laboratory, 1988; incorporated herein by reference).

In addition to polypeptides, antigens of the invention may be peptides corresponding to one or more antigenic determinants of the HIV envelope proteins of the present invention. Thus, it is contemplated that detection of an HIV antibody may be accomplished with an HIV envelope antigen that is a peptide or polypeptide.

Such peptides should generally be at least five or six amino acid residues in length and will preferably be about 10, 11, 12, 13, 14, 15, 16, 17,.18, 19, 20, 25 or about 30 amino acid residues in length, and may contain up to about 35-100 residues. For example, these peptides may comprise a HIV gp41 antigen sequence, such as 5, 6, 7, 8, 9, 10, 11, 12, 13, 14, 15, 16, 17, 18, 19, 20, 21, 22, 23, 24, 25, 30, 35, 40, 45, and 50 or more contiguous amino acids from SEQ ID NO:2. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

U.S. Pat. No. 4,554,101, incorporated herein by reference, teaches the identification and preparation of epitopes from primary amino acid sequences on the basis of hydrophilicity. Through the methods disclosed in Hopp, one of skill in the art would be able to identify epitopes and/or antigens from within an amino acid sequence such as the HIV gp41 sequence disclosed herein in as SEQ ID NO:2, 3, 4, 5, 6, 7, 8, 9, 10, 11, or 12, or amino acids 511-856 of SEQ ID NO:1.

Numerous scientific publications have also been devoted to the prediction of secondary structure, and to the identification of epitopes, from analyses of amino acid sequences (Chou & Fasman, 1974a, b; 1978a, b; 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PepPlot® (Brutlag et al., 1990; Weinberger et al., 1985), and other new programs for protein tertiary structure prediction (Fetrow & Bryant, 1993). Another commercially available software program capable of carrying out such analyses is MacVector (IBI, New Haven, Conn.).

In further embodiments, major antigenic determinants of an HIV envelope polypeptide may be identified by an empirical approach in which portions of the gene encoding an HIV envelope protein are expressed in a recombinant host, and the resulting proteins tested for their ability to elicit an immune response. Alternatively all or part of HIV envelope proteins from different subtypes or clades may be tested. A range of peptides lacking successively longer fragments of the C-terminus of the protein can be assayed as long as the peptides are prepared to retain their structure as it would be in a native polypeptide. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

6. Immunodetection Methods

As discussed, in some embodiments, the present invention concerns immunodetection methods for binding, purifying, removing, quantifying and/or otherwise detecting HIV antibodies in a sample, particularly HIV early antibodies, using substantially nondenatured or nondenatured HIV envelope antigens, such as gp41 and/or gp160. The samples may be any biological fluid or tissue from a patient. The sample may be placed on a nonreactive surface such as a plate, slide, tube, or other structure that facilitates in any way the screening of the sample for HIV antibodies. While samples may be individually screened, large numbers of samples may be screened, such as for detecting contamination in blood bank samples.

Immunodetection methods include enzyme linked immunosorbent assay (ELISA), radioimmunoassay (RLIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al., 1999; Gulbis et al., 1993; De Jager et al., 1993; and Nakamura et al., 1987, each incorporated herein by reference.

In general, the immunobinding methods include obtaining a sample suspected of containing an HIV antibody with a composition comprising an HIV envelope antigen that is substantially nondenatured or nondenatured in accordance with the present invention, as the case may be, under conditions effective to allow the formation of immunocomplexes.

These methods include methods for purifying a antibody from organelle, cell, tissue or organism's samples. In these instances, the antigen removes the antibody component from a sample. The antigen will preferably be linked to a solid support, such as in the form of a column matrix, and the sample suspected of containing the antibody will be applied to the immobilized antigen. The unwanted components will be washed from the column, leaving the antibody immunocomplexed to the immobilized antigen to be eluted. Alternatively, sandwich versions of this assay may be employed.

The immunobinding methods also include methods for detecting and quantifying the amount of an antibody component in a sample and the detection and quantification of any immune complexes formed during the binding process. Here, one would obtain a sample suspected of containing an antibody and contact the sample with an antigen, and then detect and quantify the amount of immune complexes formed under the specific conditions.

In terms of antigen detection, the biological sample analyzed may be any sample that is suspected of containing an antibody, such as, for example, a tissue section or specimen, a homogenized tissue extract, a cell, an organelle, separated and/or purified forms of any of the above antibody-containing compositions, or even any biological fluid that comes into contact with the cell or tissue, including blood and/or serum.

Contacting the chosen biological sample with the antigen under effective conditions and for a period of time sufficient to allow the formation of immune complexes (primary immune complexes) is generally a matter of simply adding the antigen composition to the sample and incubating the mixture for a period of time long enough for any antibodies present to form immune complexes with, i.e., to bind to, antigens. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antigen employed in the detection may itself be linked to a detectable label, wherein one would then simply detect this label, thereby allowing the amount of the primary immune complexes in the composition to be determined. Alternatively, the first antigen that becomes bound within the primary immune complexes may be detected by means of a second binding ligand that has binding affinity for the antigen. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

a. ELISAs

As detailed above, immnunoassays, in their most simple and/or direct sense, are binding assays. Certain preferred immunoassays are the various types of enzyme linked immunosorbent assays (ELSAS) and/or radioimmunoassays (RI) known in the art. Immunohistochemnical detection using tissue sections is also particularly useful. However, it will be readily appreciated that detection is not limited to such techniques, and/or western blotting, dot blotting, FACS analyses, and/or the like may also be used.

Turning first to immunoassays, in their most simple and direct sense, preferred immunoassays of the invention include the various types of enzyme linked imm-unosorbent assays (ELISAS) known to the art. However, it will be readily appreciated that the utility of the gp160 preparations described herein are not limited to such assays, and that other usefuil embodiments include RIAs and other non-enzyme linked antibody binding assays or procedures.

In some embodiments of the ELISA assay, native gp41 or gp160 or appropriate peptides incorporating gp41 or gp160 antigen sequences are immobilized onto a selected surface, preferably a surface exhibiting a protein affinity such as the wells of a polystyrene microtiter plate. After washing to remove incompletely adsorbed material, one will desire to bind or coat a nonspecific protein such as bovine serum albumin (BSA), casein, solutions of milk powder, gelatin, PVP, superblock, or horse albumin onto the well that is known to be antigenically neutral with regard to the test antisera. This allows for blocking of nonspecific adsorption sites on the immobilizing surface and thus reduces the background caused by nonspecific binding of antisera onto the surface. Following an appropriate coating period (for example, 3 hours), the coated wells will be blocked with a suitable protein, such as bovine serum albumin (BSA), casein, solutions of milk powder, gelatin, PVP, superblock, or horse albumin, and rinsed several times (e.g., 4 or 5 times) with a suitable buffer, such as PBS. The wells of the plates may then be allowed to dry, or may instead be used while they are still wet.

After binding of antigenic material to the well, coating with a non-reactive material to reduce background, and washing to remove unbound material, the immobilizing surface is contacted with the antisera or clinical or biological extract to be tested in a manner conducive to immune complex (antigen/antibody) formation. Such conditions preferably include diluting the antisera with diluents such as BSA, bovine gamma globulin (BGG) and phosphate buffered saline (PBS)/Tween. These added agents also tend to assist in the reduction of nonspecific background. The layered antisera is then allowed to incubate for from 1 to 4 hours, at temperatures preferably on the order of 20° to 25° C. Following incubation, the antisera-contacted surface is washed so as to remove non-immunocomplexed material. A preferred washing procedure includes washing with a solution such as PBS/Tween, or borate buffer.

Following formation of specific immunocomplexes between the test sample and the bound antigen, and subsequent washing, the occurrence and even amount of inununocomplex formation may be determined by subjecting same to a second antibody having specificity for the first. Of course, im that the test sample will typically be of human origin, the second antibody will preferably be an antibody having specificity in general for human IgG, IgM or IgA. To provide a detecting means, the second antibody will preferably have an associated enzyme that will generate a color development upon incubating with an appropriate chromogenic substrate. Thus, for example, one will desire to contact and incubate the antisera-bound surface with a urease, alkaline phosphatase, or peroxidase-conjugated anti-human IgG for a period of time and under conditions which favor the development of immunocomplex formation (e.g., incubation for 2 hours at room temperature in a PBS-containing solution such as PBS-Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethylbenzthiazoline-6-sulfonic acid (ABTS) and H₂O₂, in the case of peroxidase as the enzyme label. Quantification is then achieved by measuring the degree of color generation, e.g., using a visible spectra spectrophotometer.

In each of the microtiter wells will be placed about 10 μl of the test patient sample along with about 90 μl of reaction buffer (e.g., PBS with about 1% digitonin or other mild protein solubilizing agent). Control wells of the ELISA plate will include normal sera (human sera without early anti-HIV antibody), early anti-HIV antibody collected from HIV patient subjects who had not sero-converted as assessed using Western blot, and late anti-HIV antibody obtained from patients that have seroconverted using conventional anti-HIV antibody detection techniques.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

b. Assay Plates

In some embodiments, the wells of the assay plates may first be coated with an anti-gp41 and/or anti-gp160 antibody. This would immobilize HIV gp160 antigen to the plastic in the presence of a mild solubilizing buffer, such as from about 0.1% to about 10% digitonin (particularly about 1% digitonin). Such an approach is particularly efficacious in preparing assay plates with wells made of plastic.

The assay plates in other embodiments of the invention comprise a multiplicity of microtiter wells, and in some embodiments, polystyrene microtiter wells. These wells would be coated with about 500 ng/well of the recombinant HIV envelope protein, or recombinant. HIV antigen or HIV-infected whole cells or cell lysates thereof.

c. Immunohistochemistry

The antigens of the present invention may also be used in conjunction with both fresh-frozen and/or paraffin-embedded tissue blocks prepared for study by immunohistochemistry (IHC). (Formalin, which denatures proteins, is not used for this procedure.) HIV antibodies may be identified in this manner. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

II. Nucleic Acid Molecules

In some embodiments, the present invention concerns HIV envelope antigens prepared from genomic or recombinant nucleic acids. Some of the teachings herein pertain to the construction, manipulation, and use of nucleic acids to produce a recombinant HIV envelope antigen.

A. Polynucleotides Encoding HIV Envelope Antigens

The present invention concerns polynucleotides, isolatable from cells, that are free from total genomic DNA and that are capable of expressing all or part of a protein or polypeptide. The polynucleotide may encode a peptide or polypeptide containing all or part of an HIV envelope amino acid sequence or may encode a peptide or polypeptide having an HIV envelope antigen sequence. Recombinant proteins can be purified from expressing cells to yield nondenatured proteins or peptides.

As used herein, the term “DNA segment” refers to a DNA molecule that has been isolated free of total genomic DNA of a particular species. Therefore, a DNA segment encoding a polypeptide refers to a DNA segment that contains wild-type, polymorphic, or mutant polypeptide-coding sequences yet is isolated away from, or purified free from, total mammalian or human genomic DNA. Included within the term “DNA segment” are recombinant vectors, including, for example, plasmids, cosmids, phage, viruses, and the like.

As used in this application, the term “HIV envelope protein polynucleotide” refers to an HIV envelope protein-encoding nucleic acid molecule that has been isolated free of total genomic nucleic acid. Therefore, a “polynucleotide encoding an HIV envelope antigen” refers to a DNA segment that contains all or part of HIV envelope polypeptide-coding sequences isolated away from, or purified free from, total viral genomic DNA.

It also is contemplated that a particular polypeptide from a given species may be represented by natural variants that have slightly different nucleic acid sequences but, nonetheless, encode the same protein (see 2 above).

Similarly, a polynucleotide comprising an isolated or purified gene refers to a DNA segment including, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, which may be contiguous nucleotides encoding any length of contiguous amino acids of SEQ ID NO:1, SEQ ID NO:2, or any of SEQ ID NO:3-12.

In particular embodiments, the invention concerns isolated DNA segments and recombinant vectors incorporating DNA sequences that encode a HIV envelope antigen polypeptide or peptide, such as all or part of gp41, gp120 or gp160, which includes within its amino acid sequence a contiguous amino acid sequence in accordance with, or essentially corresponding to a native polypeptide. Thus, an isolated DNA segment or vector containing a DNA segment may encode, for example, a gp41 antigen that is capable of binding to an HIV antibody. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

Encompassed by certain embodiments of the present invention are DNA segments encoding relatively small peptides, such as, for example, a peptide comprising all or part of an HIV envelope antigen (including at least one epitope) of any subtype or clade.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless. of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated gp41 polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular gene, such as a gp41 gene of a particular subtype. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides. Such sequences may arise as a consequence of codon redundancy and flnctional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein ill vilo to a subject given the protein, or to increase the efficacy of any treatment involving the protein.

The sequence of an HIV gp41 polypeptide will substantially correspond to a contiguous portion of that shown in SEQ ID NO:2 and have relatively few amino acids that are not identical to, or a biologically functional equivalent of, the amino acids shown in SEQ ID NO:2. The term “biologically functional equivalent” is well understood in the art and is further defined in detail herein to include an ability to bind or be recognized by a specific HIV antibody.

Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:2 will be sequences that are “essentially as set forth in SEQ ID NO:2.”

In certain other embodiments, the invention concerns isolated DNA segments and recombinant vectors that include within their sequence a contiguous nucleic acid sequence from that shown in SEQ ID NO:2. This definition is used in the same sense as described above and means that the nucleic acid sequence substantially corresponds to a contiguous portion of that shown in SEQ ID NO:2 and has relatively few codons that are not identical, or functionally equivalent, to the codons of SEQ ID NO:2. The term “functionally equivalent codon” is used herein to refer to codons that encode the same amino acid, such as the six codons for arginine or serine, and also refers to codons that encode biologically equivalent amino acids. See Table 3 below, which lists the codons preferred for use in humans, with the codons listed in decreasing order of preference from left to right in the table (Wada et al., 1990). Codon preferences for other organisms also are well known to those of skill in the art (Wada et al., 1990, included herein in its entirety by reference).

TABLE 3
Preferred Human DNA Codons

| | |
|---|---|
| Amino Acids | Codons |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT |
| | | | CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA |
| | | | CGA CGT |
| Serine | Ser | S | AGC TCC TCT AGT |
| | | | TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

The various probes and primers designed around the nucleotide sequences of the present invention may be of any length. By assigning numeric values to a sequence, for example, the first residue is 1, the second residue is 2, etc., an algorithm defining all primers can be proposed:
n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It also will be understood that this invention is not limited to the particular nucleic acid encoding amino acid sequences of SEQ ID NO:1, SEQ ID NO:2, or any of SEQ ID NO:3-12. Recombinant vectors and isolated DNA segments may therefore variously include the HIV envelope antigen-coding regions themselves, coding regions bearing selected alterations or modifications in the basic coding region, or they may encode larger polypeptides that nevertheless include HIV envelope antigen-coding regions or may encode biologically functional equivalent proteins or peptides that have variant amino acids sequences.

The DNA segments of the present invention encompass biologically functional equivalent HIV envelope antigen proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein.

1. Vectors

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1996, both incorporated herein by reference. In addition to encoding a modified polypeptide such as modified gp41 or gp160, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

Vectors may include a “promoter,” which is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that fimctions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary ill vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

2. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

3. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVIROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

III. Kits

In yet another aspect of the invention, a kit is envisioned for early anti-HIV antibody detection. In some embodiments, the present invention contemplates a diagnostic kit for detecting early anti-HIV antibodies and human immunodeficiency virus infection. The kit comprises reagents capable of detecting the early anti-HIV antibody immunoreactive with the native or recombinant HIV antigens described here. Reagents of the kit include at least one HIV envelope antigen, such as all or part of gp41 and/or gp160, and any of the following: another HIV envelope antigen, buffers, secondary antibodies or antigens, or detection reagents, or a combination thereof.

In some embodiments, the kit may also comprise a suitable container means, which is a container that will not react with components of the kit, such as an eppendorf tube, an assay plate, a syringe, or a tube. In specific embodiments, the kit comprises an array or chip on which an HIV envelope antigen is placed or fixed, such as those described in Reneke et al., 2001, which is herein incorporated by reference.

In other embodiments of the invention, in addition to comprising an HIV envelope antigen, it comprises a secondary antibody capable of detecting the early anti-HIV antibody that is immunoreactive with the recombinant HIV envelope antigen.

The HIV antigen reagent of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media, plastic beads or plates, or a microscope slide. When the reagent provided is a dry powder, the powder can be reconstituted by the addition of a suitable solvent. In yet other embodiments, the kit may further comprise a container means comprising an appropriate solvent.

In some embodiments, the kit comprises a container means that includes a volume of a second antibody, such as goat anti-human IgG or IgM conjugated with alkaline phosphatase or other anti-human Ig secondary antibody, and a second container means that includes a volume of a buffer comprising a non-denaturing solubilizing agent, such as about 1% digitonin.

The kit may in other embodiments further comprise a third container means that includes an appropriate substrate, such as PNPP for alkaline phosphatase, or 9-dianisidine for peroxidase. A fourth container means that includes an appropriate “stop” buffer, such as 0.5 m NaOH, may also be included with various embodiments of the kit.

The kit may further include an instruction sheet that outlines the procedural steps of the assay, and will follow substantially the same steps as the typical EIA format known to those of skill in the art.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Subjects and HIV-1 Detection

Patients self-identified for high-risk for HIV infection were recruited over a three-year period from a Dermatology clinic (37 were eventually studied). Following signing informed consent, single blood samples (10-20 ml) were collected at their first visit, plasma separated under aseptic conditions and stored at −20° until testing. Peripheral blood mononuclear cells (PBMCs) were isolated using Lymphocyte Separation Medium (Organon Teknika, West Chester, Pa.) and rinsed once in Hank's balanced salt solution (HBSS). To deplete the PBMCs of CD8⁺ lymphocytes, approximately 10⁷ cells were incubated in 5 ml of tissue culture supernatant from the OKT8 hybridoma for 1 hr at room temperature, followed by 2 rinses in HBSS, and then placed in RPMI 1640 medium containing 20% “low-tox” rabbit complement at 37° C. for 1 hr. The PBMCs were then centrifuged and replated in medium containing 15% FBS and 4 μg/ml of phytohemagluttinin. After 3 days, the medium was changed to RPMI 1640, 15% FBS, and 40 ul/ml of IL-2 (Peprotech). Cultures were maintained for 3-4 weeks, allowing any HIV present to spread, and 0.5 ml aliquots of cells and supernatant were harvested twice weekly. Aliquots were tested for the presence of HIV p24 antigen using the Coulter HIV-1 antigen capture EIA. The remaining cultured PBLs were collected at the end of the culturing period, rinsed, and extracted for DNA for nested HIV PCR assays.

HIV DNA sequences present in the PBL DNA were determined by a nested-PCR test. The MZ 13/14 outer primer pairs for gag and 25 cycles of amplification were followed by the MZ 8/9 inner primer pairs, and 30 cycles of amplification. The assay protocol was identical to the initial report of this assay (Zazzi et al., 1993). The amplified DNA products were evaluated by agarose gel electrophoresis and ethidium bromide staining.

Live-cell Indirect Immunofluorescence Assay

An indirect microinumunofluorescence assay employing retrovirus-infected live cells as antigen has been described elsewhere (Cloyd et al., 1997; Cloyd et al., 1987). After immunostaining, the cells were resuspended in PBS containing 2% formalin and analyzed in a FACScan flow cytometer. The HIV-1 isolates used have been described previously (Race et al., 1991; Cloyd et al., 1987; Cloyd et al., 1997).

HIV Antibody EIA and Western Blot (WB) Assays

All sera were tested for anti-HIV antibodies in the Sanofi HIV-1/HIV-2 EIA. Most were also tested by an Abbott EIA in the Clinical Microbiology Laboratory at UTMB. Serum samples testing positive were subsequently tested by WB in the UTMB Clinical Microbiology Laboratory.

Radioimmunoprecipitation and SDS/PAGE

H9 cells (5×10⁷), uninfected or chronically infected with HIV₂₁₃ or HIV_{AC-1}, were incubated for 4 hr at 37° in methionine-free RPMI medium containing 30 uCi/ml [³⁵S]methionine (“Translabel”, Amersham). The cells were then washed and lysed in cold detergent solutions, and used in overnight imnmunoprecipitation assays analyzed by 10% SDS-PAGE, as described elsewhere (Race et al., 1991; Cloyd et al., 1987; Cloyd et al., 1997). The following different lysing solutions were used:
(1) 0.1% octylglucoside in 50 mM Tris-HCL pH 7.4, 30 mM NaCl, 10 mM glucose, 1 mM EDTA.
(2) 1 mM CHAPS in 50 mM Tris-HCl pH 7.4, 0.32 M sucrose, 1 mM EDTA, 0.1 mM PMSF
(3) 0.5% NP40 in TNE
(4) 20 mM digitonin in TNE
(5) 0.1% deoxycholate in TNE

Since gp41 has only a few methionines and is not labeled very well by [³⁵S] methionine, in some instances, the cells were not metabolically labeled and only lysed in various detergents. Briefly, the cells were lysed at 2×10⁷ cells/ml of lysis buffer (10 mM Tris, Ph 7.4, 130 mM NaCl, 10 mM NaF, 10 mM NaPi, 10 mM NaPPi) for one hour on ice in the presence of different detergents (2% NP40, 20 mM Digitonin, 13 mM CHAPS, 0.1% Deoxycholate or 1% SDS). The lysates were precleared by incubating with normal human sera overnight on ice. Washed Panosorbin cells (Calbiochem, San Diego, Calif.) were added into the lysates to capture the antigen-antibody complexes. The cleared supemant was then reacted with either normal (NS), “early” anti-HIV-positive (Western blot-negative), or Western blot-positive sera at 4° C. for overnight. Pansorbin cells were added and captured antibody-antigen complexes were eluted by boiling in the presence of SDS. After resolving the samples by 4-20% polyacrylamide gradient gel electrophoresis, proteins were transferred onto a nitrocellulose membrane. The presence of HIV envelope proteins were detected by Western blot using anti-gp41 and anti-gp120 monoclonal antibodies.

Native gp160 EIA

Purified native gp160 derived from soluble native gp160 produced from HIV-1_{IIIB}-infected H9 cells was obtained from Advanced Biotechnologies (Maryland, USA). Native gp160 was coated onto flat-bottom PRO-BIND EIA plates Falcon) at 50 ng/well, incubated at 37° C. for one hour followed by 4° C. for overnight. For EIA with denatured gp160, the same native gp160 was boiled for 3 minutes and cooled quickly before coating onto the plates. Detection of bound antibodies utilized biotinylated native gp160, followed by addition of streptavidin conjugated with horseradish peroxidase (1:1000 dilution; BD Pharmingen). Substrate (tetramethylbenzidine) was added for 5 min, and the color was read at a wavelength of 450/630 after stopping the reaction with 2N H₂SO₄. Three BBI seroconversion panels were tested in the native gp160 EIA at 1:4 dilution. Over 30 sera or plasma samples from low-risk University personnel were used side by side as controls at the same dilution. Duplicates of each sample were used and every test was repeated at least twice. The cutoff value was defined as two times the O.D. obtained with 24 random low-risk personnel samples. Results were expressed as signal to cutoff ratio (s/c) and s/c ratio higher than 1 is considered reactive.

### Example 2

### Identification of More High-Risk WB-Negative Subjects Possessing “Early HIV Ab” in Their Serum

To further study these antibodies, serum samples were obtained from more than the four that had been originally identified (Race et al., 1991). Patients attending a dermatology clinic were queried as to their risk for HIV infection and 37 high-risk subjects were eventually identified over a three-year period. None of these individuals were known to be HIV-positive, but all revealed high-risk histories and presented with dermatological problems. Blood was drawn from each subject, following informed consent, and tested for serum anti-HIV antibodies using the nondenaturing live-cell IFA, as well as by commercial EIAs (Abbott or Sanofi). After IFA staining of the live cells with serial dilutions of subject's plasma, they were examined by flow cytometry. Eight subjects showed reactivity in the live-cell IFA, while non-reactive in Western blot. The serum antibody IFA titers of the 8 WB-negative patients were lower (Table 4) than the titers in the sera of 17 patients who were found to be WB-positive (320-5,000), reproducing results from previous studies (Race et al., 1991; Cloyd et al., 1987; Cloyd et al., 1997). None of these sera reacted with uninfected cells, indicating that the antibodies present were reacting to either HIV proteins or HIV-induced cellular proteins. Sera from 74 low-risk normal subjects (low-risk university personnel) have been similarly analyzed over many years, and were always negative for both infected and uninfected target cells at dilutions of 1:15 or higher.

### Example 3

### Testing the Patients Possessing “Early Anti-HIV Antibodies” for HIV

It was crucial to determine whether the WB-negative individuals who possessed “early HIV Ab” were actually HIV-infected. CD8 T-cell-depleted PBL cultures from these individuals were tested by p24 antigen-capture EIA over 4-weeks of culture, and 4 of 8 were found to contain HIV p24 (Table 5). DNAs extracted from the PBLs of the four p24-negative subjects at the end of the culturing period were positive by nested PCR for HIV gag sequences. As controls, 5 high-risk individuals who were negative in live-cell IFA were also tested, and all were found to be negative for HIV by p24 antigen-capture EIA of supernatants from cultures of their PBLs and HIV nested PCR amplification of their PBL DNA (Table 5). Thus, the presence of “early HIV Ab” in the sera of EIA- and/or Western blot-negative individuals tested to date correlates 100% with the presence of HIV in their PBLS.

### Example 4

### The Specificity of ““Early IV Antibodies””

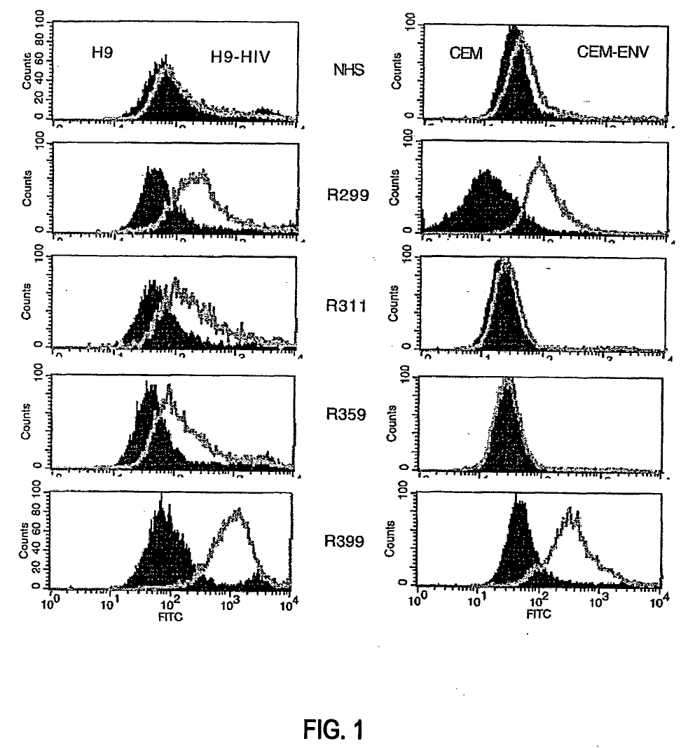

A previous radioimmunoprecipitation and SDS-PAGE study demonstrated that the Ab in the serum of one early infected, “early Ab” -positive subject reacted only with HIV gp160 and not gp120 (Race et al., 1991). Thus the sera from the current subjects were tested in live-cell IFA against cell lines expressing only HIV envelope. These are CEM T-cells, which express the envelope genes from HIV₂₁₃, HIV_{AC-1}, or HIV_{C}, (Keller et al., 1996), three viruses used for testing the presence of HIV Ab (Race et al., 1991; Cloyd et al., 1987; Cloyd et a!, 1997). FIG. 1 shows flow cytometry results of 3 sera, demonstrating that some did not react with cells expressing only envelope protein, although they did react to cells replicating whole HIV (FIG. 1A). Sera from R311 and R359 did not react with any of the three HIV envelope-expressing lines (FIG. 1B), but R299 did react. Table 6 summarizes the results of testing three of the sera originally reported (Race et al., 1991) and the eight new sera, showing that eight of eleven reacted with envelope proteins. However, it appears that the Ab in some subjects are reacting with protein(s) other than HIV envelope. However, before such studies were performed, it was first decided what was the best way to solubilize the HIV-infected cells in order to retain reactivity with these Ab, since they were very likely binding to conformational epitopes. “early HIV antibodies” reacted readily with HIV proteins solubilized in digitonin and NP40, but poorly or not at all when solubilized in deoxycholate, CHAPS, octylglucoside, or SDS.

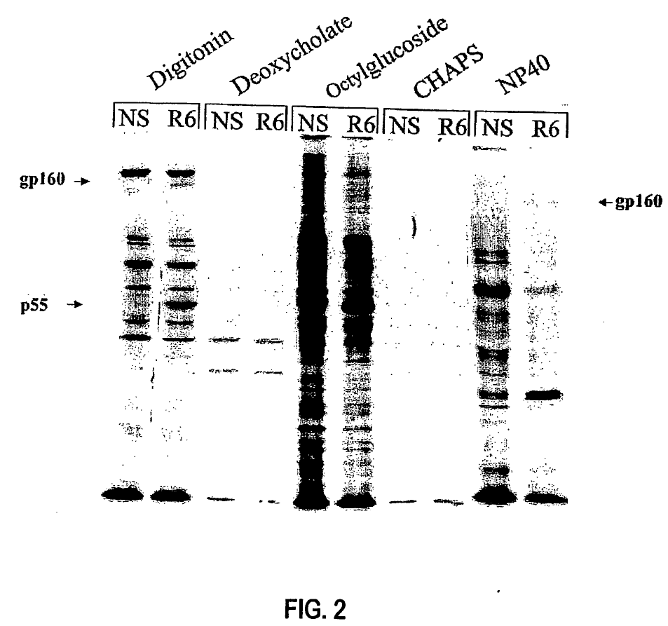

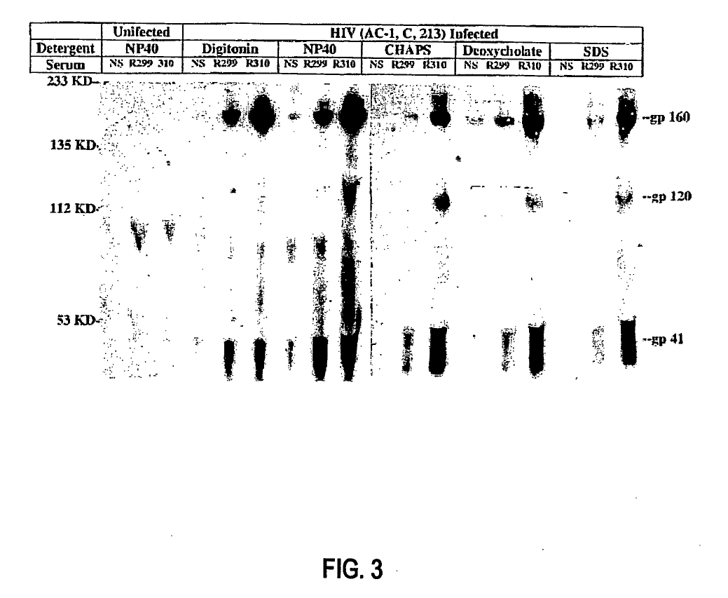

To identify what HIV proteins the antibodies in these sera reacted to, radioimmunoprecipitation (RIP) of detergent lysates of HIV-infected H9 cells metabolically labeled with [³⁵S] methionine and cystine were used. The infected cells were solubilized in digitonin, which is one of the least denaturing detergents and is often used to solubilize protein complexes, as well as in NP40, deoxycholate, octylglucoside, or CHAPS. FIG. 2 shows the reactivity of one of the previously described patients (R6) sera (Race et al., 1991) for proteins solubilized in the various detergents. A protein of approximately 160 Kd was precipitated from infected cells solubilized in digitonin and NP40, but not when solubilized in the other detergents. In addition, a protein of around 55 kd was precipitated from the digitonin lysate, but not from the other lysates. This strongly indicates that the epitopes recognized by the antibody are likely confornational in nature since they were very sensitive to denaturation. Control EIA and Western blot-positive sera showed that all 4 lysates contained other HIV proteins in addition to gp160 and p55, but gp41 was present in very small detectable quantity. Identical results were obtained when R6 and R23 sera were repeated. In order to test if this conformation-dependent reactivity of “early HIV Abs” is universal, R299 was also analyzed using immunoprecipitation again, but with a Western blot format for readout. Uninfected or HIV-infected H9 cells were solubilized in digitonin, NP40, deoxycholate, octyglucoside, CHAPS or SDS. These lysates were then incubated with normal serum (NS), “early HIV Ab”-containing serum (R299) or an HIV Western blot-positive serum (R310), and the immunoprecipitates were resolved on SDS-PAGE followed by Western blot analysis with anti HIV-gp41 (Chessie 8) and gp120 (Chessie 6) specific mAb. FIG. 3 shows the results. No proteins were precipitated from uninfected cells with R299 and WB-positive control sera R310. However, when mild detergents such as Digitonin, and NP40, were used, maximum levels of HIV envelope protein gp160 as well as gp41, but not gp120 were precipitated with R299, while the WB-positive serum also precipitated gp120. When stronger detergents CHAPS and SDS were used, the majority of reactivity with R299 was lost.

To further substantiate that this 160 Kd protein was HIV gp160 and not a 160 Kd cellular protein induced by HIV infection, NP40-solubilized HIV-infected H9 cell lysate was first pre-cleared with an anti-gp120 monoclonal antibody (F105) before immunoprecipitation with the sera from R6 and R23. Pre-clearing eliminated the 160 Kd protein from the lysate that the “early HIV Ab” inmmunoprecipitated, demonstrating that this Ab was recognizing HIV gp 160.

### Example 5

### Use of an EIA Employing Non-denatured gp160

A third generation EIAs using purified native, non-denatured gp160 was next developed to detect these “early HIV Ab”. Table 7 shows the results of using this EIA employing HIV gp160 coated either in its native form or denatured by boiling. The ODs obtained with “early HIV Ab-”-containing sera of subjects R299, BR22, and R400 on native gp160 demonstrates reactivity (>1). However, using plates coated with denatured gp160, none of the sera were reactive. To show that both plates contained similar amount of gp160, a serum positive in WB for HIV Ab was used and similarly high ODs were observed on both plates. This further confirms that the “early HIV Ab” present in early infected individuals react with only conformational, not primary, amino acid epitopes of gp160.

### Example 6

### “Early HIV antibodies” are Present Much Earlier than Seroconversion in Current EIA, Antigen and RT-PCR Assays

The above results, as well as our previous studies (Race et al., 1991), clearly show that antibodies reacting with conformational epitopes of cell surface-expressed HIV gp41 are present in early-infected individuals' sera prior to antibodies that react in denatured antigen (EIA and WB) assays. An important remaining question concerns how much earlier, before seropositivity in the latter tests, is this “early HIV antibody” present? To address this question, five BBI seroconversion panels were tested in our new third generation native gp160 EIA (for seroconversion panels, see BBI Catalog in Appendix 1). Tables 8a-e show the results of these seroconversion panels. In Table 8a, panel PRB 931, the initial bleed was negative in our test, but the serum obtained two days later was positive as well as later bleeds. However, not until day 15 did both the Coulter HIV antigen and Roche RT-PCR tests start to become positive. Thirteen days later, a commercial EIA (Abbott HIV-1/2) showed positivity. Thus, early antibodies were present in this panel thirteen days prior to positivity in the Coulter HIV antigen and Roche RT-PCR tests and approximately 26 days earlier than positivity for antibodies reactive in the commercially available denatured antigen EIAs. In the second panel PRB923 (Table 8b), the first bleed and subsequent bleeds up to day 30 were positive in our test, but the Ab titers continually decreased. On day 35 the native gp160 EIA became negative and the plasma became positive for HIV RNA by the Roche RT-PCR test. It is very likely that the continued decreases in “early HIV Ab” over time were due to slowly increasing virus loads, which likely absorbed the antibodies and kept them from reacting in the assay. Thus, scoring for “early HIV Abs” allowed detection of HIV infection approximately 35 days earlier than scoring for HIV RNA by the Roche viral load test, 37 days earlier than the most sensitive HIV antigen test and 47 days earlier than the Abbott HIV-1/2 antibody test in this panel. In the third panel, PRB932 (Table 8c), the first bleed was positive in our test, which was 27 days earlier than positivity in both the Roche RNA and the Abbott HIV-1/2 Ab test. Two more panels are shown in Tables 8d and 8e, further confirming our observations.

Table 9 summarizes the results from all 3 panels in native gp160 antibody assays in comparison to standard reference HIV antibody, antigen and RNA assays. In summary, native gp160 assays, detected HIV infection between 13-44, 13-47 and 26-47 days earlier then the best HIV RNA, antigen, and antibody assays, respectively.

“Early HIV Ab” was then tested in a clinical situation. Serum samples from an HIV needle-stick case were provided by CDC and were tested by live-cell IFA and our native gp160 EIA. Serum taken 3 weeks after the exposure was commercial EIA-negative, but positive in both the native gp160 EIA and live-cell IFA (Table 10). By 4 months post-exposure, the serum was positive in all tests (Table 10). Again, this case further demonstrated that a test for “early HIV Ab” would allow earlier diagnosis of infection.

TABLE 4
Live-Cell IFA Serum Antibody Titers of WB-Negative Subjects
Reciprocal of serum dilution that stained 50% of
H9 cells infected with these HIV strains

| Subject | Uninf | 213 | AC1 | C | RF | MN | III_{B} |
|---|---|---|---|---|---|---|---|
| R291 | — | 30 | — | 30 | — | 60 | 30 |
| R299 | — | 60 | 75 | 60 | 40 | 40 | 100 |
| R311 | — | 30 | 40 | 60 | 70 | 30 | 50 |
| R312 | — | 100 | 80 | 120 | 50 | 100 | 80 |
| R328 | — | 30 | 50 | 30 | — | 30 | — |
| R343 | — | 80 | 30 | — | — | 30 | 80 |
| R359 | — | 60 | 30 | 50 | — | — | 30 |
| R400 | — | 50 | 80 | 60 | — | — | 30 |

TABLE 5
HIV Detection in PBLs of High-Risk WB-Negative,
Live-Cell IFA-Positive Subjects

| | CD4 PBL Culture | |
|---|---|---|
| Subject | P24 Antigen Capture EIA | HIV Nested PCR |

| | | |
|---|---|---|
| (IFA-Pos): | | |
| R291 | + | + |
| R299 | − | + |
| R311 | − | + |
| P312 | − | + |
| R328 | + | ND |
| P343 | + | ND |
| P359 | + | ND |
| R400 | − | + |
| (IFA-Neg): | | |
| P239 | − | − |
| P285 | − | − |
| P296 | − | − |
| P297 | − | − |
| P303 | − | − |

ND = not determined

TABLE 6
Summary of Live-Cell IFA Testing of EIA- and/or WB-negative Sera

| | | | Live-Cell IFA | |
|---|---|---|---|---|
| Subject | EIA | WB | Complete HIV | ENV Only |
| R6 | — | — | + | + |
| R23 | — | — | + | + |
| R78 | — | — | + | |
| P291 | — | — | + | + |
| P299 | — | — | + | + |
| P311 | — | — | + | + |
| P312 | — | — | + | + |
| P328 | + | — | + | — |
| P343 | + | — | + | — |
| P359 | — | — | + | — |
| R400 | — | — | + | + |

TABLE 7
EIA Results Using Native or Denatured HIV gp160-Coated Plates.
Signal to cutoff⁽¹⁾ ratios with serum dilutions of 1:4

| Serum | | |
|---|---|---|
| (No. tested) | Native gp160 | Denatured gp160 |
| NHS (5) | 0.50 | 0.50 |
| R299 | 2.3 | 0.64 |
| HR22 | 5.9 | 0.72 |
| R400 | 4.7 | 0.59 |
| R292 | 33 | 40 |
| (WB+) | | |

⁽¹⁾Cutoff = 2 x the O.D. obtained with normal human sera (NHS).

TABLE 8
Comparisons of Native GP160 EIA with the most sensitive commercial
Antibody, Antigen, RNA and Western blot assay on HIV-1
Seroconversion Panels

| | | | | | |
|---|---|---|---|---|---|
| | Native | | Coulter | | |
| Days | GP 160 | Abbott | HIV | Roche | Ortho/Cambridge |
| Since | EIA | HIV ½ | Antigen | HIV RNA | Western Blot |
| 1^{st} Bleed | UTMB | BBI^{(a)} | BBI^{(a)} | BBI^{(a)} | BBI^{(a)} |

TABLE 8a
BBI PRB931

| | | | | | |
|---|---|---|---|---|---|
| 0 | 0.72^{(b)} | 0.1 | 0.1 | BLD | NEG |
| 2 | 2.17 | 0.1 | 0.1 | BLD | NEG |
| 7 | 4.94 | 0.1 | 0.1 | BLD | NEG |
| 9 | 1.62 | 0.1 | 0.2 | BLD | NEG |
| 15 | 6.14 | 0.1 | 1.2 | 3 x 10⁴ | NEG |
| 28 | 9.21 | 6.0 | 11.7 | 2 x 10⁶ | NEG |
| 33 | 12.12 | >18.7 | 17.5 | 1 x 10⁶ | POS |
| 35 | 11.92 | >18.7 | 9.5 | 5 x 10⁶ | POS |
| 42 | 13.29 | >18.7 | 8.3 | 1 x 10⁶ | POS |

TABLE 8b
BBI PRB923

| | | | | | |
|---|---|---|---|---|---|
| 0 | 13.43^{(b)} | 0.1 | 0.4 | NEG | NEG |
| 7 | 6.32 | 0.1 | 0.4 | NEG | NEG |
| 12 | 6.49 | 0.1 | 0.5 | NEG | NEG |
| 14 | 2.08 | 0.1 | 0.4 | NEG | NEG |
| 28 | 4.29 | 0.1 | 0.4 | NEG | NEG |
| 30 | 2.57 | 0.1 | 0.4 | NEG | NEG |
| 35 | 0.51 | 0.1 | 0.4 | POS | NEG |
| 37 | 1.88 | 0.1 | 1.0 | POS | NEG |
| 47 | 1.04 | 1.4 | >23.3 | POS | NEG |
| 84 | 8.50 | 7.8 | 0.4 | POS | POS |
| 86 | 7.21 | 7.6 | 0.4 | POS | POS |
| 145 | 22.88 | >17.2 | 0.4 | POS | POS |
| 161 | 23.21 | >17.2 | 0.5 | POS | POS |

TABLE 8c
BBI PRB932

| | | | | | |
|---|---|---|---|---|---|
| 0 | 2.71^{(b)} | 0.1 | 0.1 | BLD⁽²⁾ | NEG |
| 3 | 0.63 | 0.1 | 0.0 | BLD | NEG |
| 13 | 1.08 | 0.2 | 0.0 | BLD | NEG |
| 27 | 0.65 | 1.1 | 18.6 | 4 X 10⁵ | NEG |
| 34 | 6.24 | 12.5 | 17.8 | 5 X 10⁵ | POS |
| 50 | 1.06 | 1.7 | 5.4 | 2 X 10⁵ | POS |
| 78 | 0.76 | 0.6 | 9.2 | 4 X 10⁵ | POS |
| 163 | 11.37 | 2.1 | 4.8 | 3 X 10⁵ | POS |
| 194 | 8.04 | 8.6 | 2.9 | 7 X 10⁴ | POS |

TABLE 8d
BBI PRB925

| | | | | | |
|---|---|---|---|---|---|
| 0 | .55^{(b)} | 0.1 | 0.5 | NEG | NEG |
| 10 | .40 | 0.1 | 0.5 | NEG | NEG |
| 18 | .65 | 0.1 | 0.5 | NEG | NEG |
| 22 | .40 | 0.1 | 0.5 | NEG | NEG |
| 44 | 4.76 | 11.8 | 3.7 | POS | IND |
| 49 | 5.80 | 9.9 | 2.6 | POS | IND |

TABLE 8e
BBI PRB935

| | | | | | |
|---|---|---|---|---|---|
| 0 | .37^{(b)} | 0.1 | 0.0 | BLD^{(a)} | NEG |
| 10 | .46 | 0.1 | 0.0 | BLD | NEG |
| 16 | .38 | 0.1 | 0.0 | BLD | NEG |
| 21 | .33 | 0.1 | 0.0 | BLD | NEG |
| 24 | .38 | 0.1 | 0.0 | 4 x 10³ | NEG |
| 28 | .34 | 0.1 | 6.8 | 4 x 10⁶ | NEG |
| 43 | 1.65 | 3.9 | 5.7 | 5 x 10⁶ | POS |

Note: Underlined data points indicate first reactivity of an individual assay for the serum series.
BLD, below detection; NEG, negative; POS, positive; IND, indeterminate.
^{(a)}Data obtained from BBI is taken from their data sheet, and is expressed as a signal/cutoff ratio where 1 or greater is considered positive.
^{(b)}Results expressed as signal/cutoff ratio with cutoff defined as an OD 2x that of the mean OD with 24 normal control sera tested in duplicate. Signal/cutoff ratio greater than 1 is considered reactive.

TABLE 9
Comparison of the performance of Native gp160 EIA_{IIIb,}
HIV-1/HIV-2 3^{rd} Generation Plus EIA, p24 Ag detection,
HIV-1 RNA RT-PCR, and Western blot in BBI seroconversion panels.
Bleeding day with first positive result in:

| | | HIV | | | |
|---|---|---|---|---|---|
| | Native | -1/HIV-2 | | | |
| Seroconversion | gp160 | 3^{rd} gene | Pry | | Western |
| Panel | EIA_{IIIb} | EIA | Ag⁽¹⁾ | RT-PCR⁽¹⁾ | Blot⁽¹⁾ |
| BBI AF 931 | 2 | 28 | 15 | 15 | 33 |
| BBI AG 932 | 0 | 27 | 27 | 27 | 34 |
| BB1 W 923 | 0 | 47 | 47 | 35 | 84 |

⁽¹⁾Data from BBI
ND, not determined.

TABLE 10
Timing of Early Seroconversion in a Health Care
Worker Exposed to HIV+ Blood Via Needle-Stick.

| | | | HIV antibody detected by | | | |
|---|---|---|---|---|---|---|
| Serum | Time | Commercial | | L | | |
| Sample | After | ELA | | | | |
| | Exposure | | | | | |
| | | | H9-213 | H9-AC-1 | | |
| 40499 | 3 weeks | − | 320¹ | 160 | 40 | 2.3² |
| 542837 | 4 months | + | 1200 | 1000 | 400 | 18.8 |

¹Reciprocal of dilution of serum that fluoresced 50% of cells
²Signal to cutoff ratio where cutoff = 2 × O.D. with normal sera

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
EPA No. 320 308
EPA No. 329 822
GB Application No. 2,202,328
GB Application No. 2193095
PCT/US85/01161
PCT/US87/00880
PCT/US89/01025
PCT/US89/05040
U.S. Pat. No. 3,817,837
U.S. Pat. No. 3,850,752
U.S. Pat. No. 3,939,350
U.S. Pat. No. 3,996,345
U.S. Pat. No. 4,162,282
U.S. Pat. No. 4,196,265
U.S. Pat. No. 4,275,149
U.S. Pat. No. 4,277,437
U.S. Pat. No. 4,310,505
U.S. Pat. No. 4,366,241
U.S. Pat. No. 4,533,254
U.S. Pat. No. 4,554,101
U.S. Pat. No. 4,683,195
U.S. Pat. No. 4,683,202
U.S. Pat. No. 4,684,611
U.S. Pat. No. 4,728,575
U.S. Pat. No. 4,728,578
U.S. Pat. No. 4,737,323
U.S. Pat. No. 4,800,159
U.S. Pat. No. 4,873,191
U.S. Pat. No. 4,879,236
U.S. Pat. No. 4,883,750
U.S. Pat. No. 4,921,706
U.S. Pat. No. 4,946,773
U.S. Pat. No. 4,952,500
U.S. Pat. No. 5,054,297
U.S. Pat. No. 5,175,384
U.S. Pat. No. 5,175,385
U.S. Pat. No. 5,279,721
U.S. Pat. No. 5,302,523
U.S. Pat. No. 5,322,783
U.S. Pat. No. 5,354,855
U.S. Pat. No. 5,384,253
U.S. Pat. No. 5,399,363
U.S. Pat. No. 5,464,765
U.S. Pat. No. 5,466,468
U.S. Pat. No. 5,530,179
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,565,186
U.S. Pat. No. 5,580,859
U.S. Pat. No. 5,589,466
U.S. Pat. No. 5,591,616
U.S. Pat. No. 5,609,870
U.S. Pat. No. 5,610,042
U.S. Pat. No. 5,612,486
U.S. Pat. No. 5,616,491
U.S. Pat. No. 5,625,125
U.S. Pat. No. 5,639,457
U.S. Pat. No. 5,641,515
U.S. Pat. No. 5,656,610
U.S. Pat. No. 5,693,762
U.S. Pat. No. 5,702,932
U.S. Pat. No. 5,736,524
U.S. Pat. No. 5,739,169
U.S. Pat. No. 5,780,448
U.S. Pat. No. 5,785,970
U.S. Pat. No. 5,789,215
U.S. Pat. No. 5,801,005
U.S. Pat. No. 5,824,311
U.S. Pat. No. 5,830,880
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,225
U.S. Pat. No. 5,846,233
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,155
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,871,986
U.S. Pat. No. 5,879,703
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,925,565
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,980,912
U.S. Pat. No. 5,981,274
U.S. Pat. No. 5,994,624
U.S. Pat. No. 6,020,192
U.S. Pat. No. 6,027,727
U.S. Pat. No. 6,054,297
WO 84/03564
WO 88/10315
WO 89/06700
WO 90/07641
WO 94/09699
WO 95/06128
WO 99/18933
Abbondanzo et al., Breast Cancer Res. Treat., 16:182(#151), 1990.
Adams & Cory, Science. 281:1322-6, 1998.
Aiuti et al., Vaccine, 11:538-41, 1993.
Allred et al., Breast Cancer Res. Treat., 16:182(#149), 1990.
Almendro et al., J. Immunol., 157:5411-5421, 1996.
Ambrosini et al., Nat Med., 3:917-21, 1997.
Angel et al., Cell, 49:729, 1987b.
Angel et al., Mol. Cell. Biol., 7:2256, 1987a.
Arap et al., Cancer Res., 55:1351-1354, 1995.
Atchison and Perry, Cell, 46:253, 1986.
Atchison and Perry, Cell, 48:121, 1987.
AuBuchon, Ann Intern Med, 127:904-9, 1997.
Austin-Ward, Villaseca, Rev. Med. Chil., 126(7):838-45, 1998.
Ausubel et al., Current Protocols in Molecular Biology, (John Wiley & Sons, Inc., New York, N.Y., 1998.
Ausubel, ed., Current protocols in molecular biology, New York, John Wiley & Sons, 1996.
Baichwal and Sugden, In: Gene Transfer; Kucherlapati R, ed., New York, Plenum Press, pp. 117-148, 1986.
Bajorin et al., Proc. Annu. Meet. Am. Soc. Clin. Oncol., 7:A967, 1988.
Bakhshi et al., Cell, 41(3):899-906, 1985.
Banerji et al, Cell, 27:299, 1981.
Banerji et al., Cell, 35:729, 1983.
Bangham, et al., J Mol. Biol., 13:238-252, 1965.
Barbiano di Belgiojoso et al., J Nephrol, 11:249-54, 1998.
Bellus, J. Macromol. Sci. Pure Appl. Chem, A31(1): 1355-1376, 1994.
Benjamini, “Immunology: A Short Course,” Wiley-Liss, New York (3^{rd} ed., 1991).
Berkhout et al., Cell, 59:273, 1989.
Berzal-Herranz, A. et al., Genes and Devel., 6:129-134, 1992.
Blanar et al., EMBO J., 8:1139, 1989.
Bodine and Ley, EMBO J., 6:2997, 1987.
Bonavida et al., Int J. Oncol, 15:793-802, 1999.
Bonavida et al., Proc Nat'l Acad Sci USA. 97:1754-9, 2000.
Boshart et al., Cell, 41:521, 1985.
Bosze et al, EMBO J., 5:1615, 1986.
Braddock et al., Cell, 58:269, 1989.
Brettler et al., Blood, 80:2396-400, 1992.
Brinster et al., Proc. Nat'l Acad. Sci. USA, 82:4438-4442, 1985.
Brown et al. Breast Cancer Res. Treat., 16:192(#191), 1990.
Brown et al., Transfusion, 37:926-9, 1997.
Brutlag et al., CABIOS, 6:237-245, 1990.
Bukowski et al., Clin. Cancer Res., 4(10):2337-47, 1998.
Bulla and Siddiqui, J. Virol., 62:1437, 1986.
Busch et al., N Engl J Med, 325:1-5, 1991.
Busch et al., Transfusion, 35:91-7, 1995.
Caldas et al., Nat. Genet., 8:27-32, 1994.
Campbell and Villarreal, Mol. Cell. Biol., 8:1993, 1988.
Campere and Tilghman, Genes and Dev., 3:537, 1989.
Campo et al., Nature, 303:77, 1983.
Canfield et al., Methods in Enzymology, 189, 418-422, 1990.
Capaldi et al., Biochem. Biophys. Res. Comm., 76:425, 1977.
Carbonelli et al. FEMS Microbiol Lett. 177(1):75-82, 1999.
Cech et al., Cell, 27:487-496, 1981.
Celander and Haseltine, J. Virology, 61:269, 1987.
Celander et al., J. Virology, 62:1314, 1988.
Chandler et al., Cell, 33:489, 1983.
Chandler et al., Proc Natl Acad Sci USA. 94(8):3596-3601, 1997.
Chatterjee et al., Proc. Nat'l Acad. Sci. USA., 86:9114, 1989.
Chen and Okayama, Mol. Cell Biol., 7:2745-2752, 1987.
Cheng et al., Cancer Res., 54:5547-5551, 1994.
Cheng, et al., Investigative Radiology, vol. 22, pp. 47-55 (1987).
Chitpatima et al., Nucleic Acids Res., 16:2350, 1988.
Chou and Fasman, “Conformational Parameters for Amino Acids in Helical, β-Sheet, and Random Coil Regions Calculated from Proteins,” Biochemistry, 13(2):211-222, 1974b.
Chou and Fasman, Adv. Enzymol. Relat. Areas Mol. Biol., 47:45-148, 1978a.
Chou and Fasman, Ann. Rev. Biochem., 47:251-276, 1978b.
Chou and Fasman, Biochemistry, 13(2):222-245, 1974a.
Chou and Fasman, Biophys. J., 26:367-384, 1979.
Chowrira, B. H. et al., Biochemistry, 32:1088-1095, 1993.
Chowrira, B. H. et al., J. Biol. Chem., 269:25856-25864, 1994.
Christodoulides et al., Microbiology, 144(Pt 11):3027-37, 1998.
Chung et al., Cancer Lett., 156:185-90, 2000.
Cleary and Sklar, Proc. Nat'l. Acad. Sci. USA, 82(21):7439-43, 1985.
Cleary et al., J. Exp. Med., 164(1):315-20, 1986.
Clerici etal., J Infect Dis, 164:178-82, 1991.
Clerici et al., Jama, 271:42-6, 1994(a).
Clerici et al., Lancet, 343:1552-3, 1994(b).
ClonTech. Yeast Protocols Handbook 1997.
Cloyd and Bigner, J Clin Microbiol, 5:86-90, 1977.
Cloyd and Holt, Virology, 2:286-92, 1987.
Cloyd and Moore, Virology, 174:103-16, 1990.
Cocea, Biotechniques. 23(5):814-816, 1997.
Cohen et al., J. Cell. Physiol., 5:75, 1987.
Costa et al., Mol. Cell. Biol., 8:81, 1988.
Coupar et al., Gene, 68:1-10, 1988.
Cripe et al., EMBO J., 6:3745, 1987.
Culotta and Hamer, Mol. Cell. Biol., 9:1376, 1989.
Culver et al., Science, 256:1550-1552, 1992.
Daidone et al., Endocr Relat Cancer, 6;61-8, 1999.
Dandolo et al., J. Virology, 47:55, 1983.
Davidson et al., J. Immunother., 21(5):389-98, 1998.
De Villiers et al., Nature, 312:242, 1984.
Deamer and P. Uster, Liposomes (M. Ostro, ed.), Marcel Dekker, Inc., New York, pp. 27-52, 1983.
Dejager et al., J. Clin. Invest., 92:894-902, 1993.
Deschamps et al., Science, 230:1174, 1985.
Deveraux et al., Nature 388:300-4, 1997.
Dillman Cancer Biother. Radiophann., 14:5-10, 1999.
Doolittle et al., Methods Mol. Biol., 109:215-37, 1999.
Duckett et al., Embo J., 15:2685-94, 1996.
Eble et al., Transfusion, 32:503-8, 1992.
Edbrooke et al., Mol. Cell. Biol., 9:1908, 1989.
Edlund et al., Science, 230:912, 1985.
Eichinger et al., J Biol Chem 273(21), 12952-9, 1998.
El-Gorab et al., Biochem. Biophys. Acta, 1973, 306, 58-66, 1973.
Engelman et al., Annu Rev Biophys Biopizys Chem., 15:321-53, 1986.
Fechheimer et al., Proc. Nat'l Acad. Sci. USA, 84:8463-8467, 1987.
Fendler et al., Catalysis in Micellar and Macromolecular Systems, Academic Press, New York, 1975.
Feng and Holland, Nature, 334:6178, 1988.
Ferre, Clin Lab Med, 14:313-33, 1994.
Fetrow and Bryant, Biotech., 11:479-483, 1993.
Firak and Subramanian, Mol. Cell. Biol., 6:3667, 1986.
Fodor et al., Science, 251:767-773, 1991.
Foecking and Hofstetter, Gene, 45(1):101-5, 1986.
Folkman and Shing, J Biol Chem 267(16), 10931-10934, 1987.
Folkman and Klagsbrun, Science, 235: 442-447, 1987.
Forster and Symons, Cell, 49:211-220, 1987.
Fraley et al., Proc. Nat'l Acad. Sci. USA, 76:3348-3352, 1979.
Friedmann, Science, 244:1275-1281, 1989.
Frohman, In: PCR Protocols: A Guide To Methods And Applications, Academic Press, N.Y., 1990.
Fujise et al., J Biol Chem 275, 39458-39465, 2000.
Fujita et al., Cell, 49:357, 1987.
Fung-Leung et al., Cell, 65:443-449, 1991a.
Fung-Leung et al., J. Exp. Med., 174:1425-1429, 1991b.
Gabizon et al., Cancer Res., 50(19):6371-8, 1990.
Gerlach et al., Nature (London), 328:802-805, 1987.
Ghose and Blair, Crit. Rev. Ther. Drug Carrier Syst., 3(4):263-359, 1987.
Ghosh and Bachhawat, In: Liver diseases, targeted diagnosis and therapy using specific receptors and ligands, (Wu G, Wu C ed.), New York: Marcel Dekker, pp. 87-104, 1991.
Gilles et al., Cell, 33:717, 1983.
Gliniak et al., Cancer Res. 59:6153-8, 1999.
Gloss et al., EMBO J., 6:3735, 1987.
Godbout et al., Mol. Cell. Biol., 8:1169, 1988.
Gong et al., J Biol Chem., 272:28198-201, 1997.
Goodbourn and Maniatis, Proc. Nat'l Acad. Sci. USA, 85:1447, 1988.
Goodbourn et al., Cell, 45:601, 1986.
Gopal, Mol. Cell Biol., 5:1188-1190, 1985.
Graham and Van Der Eb, Virology, 52:456-467, 1973.
Greene et al., Immunology Today, 10:272, 1989.
Gregoriadis, ed., Drug Carriers In Biology And Medicine, pp. 287-341, 1979.
Gregoriadis, G., ed., Liposome Technology, vol. I, pp. 30-35, 51-65 and 79-107 (CRC Press Inc., Boca Raton, Fla., 1984.
Gross et al., Nucleic Acids Res., 17:8367, 1989.
Grosschedl and Baltimore, Cell, 41:885, 1985.
Guidozzi, et al., Gynecol Ozcol., 61:204-9, 1996.
Gulbis et al., Hum. Pathol., 24:1271-85, 1993.
Gupta et al., Aids, 6:143-9, 1992.
Hacia et al., Nature Genetics, 14:441-447, 1996.
Hande et al., Eur J Cancer, 34:1514-21, 1998.
Hanibuchi et al., Int. J Cancer, 78(4):480-5, 1998.
Harland and Weintraub, J. Cell Biol., 101:1094-1099, 1985.
Harlow and Lane, “Antibodies: A Laboratory Manual,” Cold Spring Harbor Laboratory, 1988.
Haseloff and Gerlach, Nature, 334:585-591, 1988.
Haslinger and Karin, Proc. Nat'l Acad. Sci. USA., 82:8572, 1985.
Hauber and Cullen, J. Virology, 62:673, 1988.
Hellstrand et al., Acta. Oncol., 37(4):347-53, 1998.
Hen et al., Nature, 321:249, 1986.
Hensel et al., Lymphokine Res., 8:347, 1989.
Hermonat and Muzyczka, Proc. Nat'l. Acad. Sci. USA, 81:6466-6470, 1984.
Herr and Clarke, Cell, 45:461, 1986.
Hirochika et al., J. Virol., 61:2599, 1987.
Hirsch et al., Mol. Cell. Biol., 10:1959, 1990.
Hogan et al., Manipulating the Mouse Embryo, Cold Spring Harbor Laboratory, Cold Spring Harbor, N.Y., 1986.
Holbrook et al., Virology, 157:211, 1987.
Hollstein et al., Science 253:49-53, 1991.
Horlick and Benfield, Mol. Cell. Biol., 9:2396, 1989.
Horwich et al. J. Virol., 64:642-650, 1990.
Huang et al., Cell, 27:245, 1981.
Hug et al., Mol Cell Biol., 8:3065, 1988.
Hui and Hashimoto, Infect. Immun., 66(11):5329-36, 1998.
Hussussian et al., Nature Genetics, 15-21, 1994.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., Proc Natl Acad Sci USA. 85(24):9436-9440, 1988.
Inouye et al., Nucleic Acids Res., 13:3101-3109, 1985.
Irie & Morton, Proc. Nat'l Acad. Sci. USA 83:8694-8698, 1986
Erie et al., “Melanoma gangliosides and human monoclonal antibody,” In: Human Tumor Antigens and Specific Tumor Therapy, Metzgar & Mitchell (eds.), Alan R. Liss, Inc., New York, pp. 115-126, 1989.
Jakobovits et al., Mol. Cell. Biol., 8:2555, 1988.
Jameel and Siddiqui, Mol. Cell. Biol., 6:710, 1986.
Jameson and Wolf, Comput. Appl. Biosci., 4(1):181-186, 1988.
Jaynes et al., Mol. Cell. Biol., 8:62, 1988.
Jehuda-Cohen et al., Proc Natl Acad Sci USA, 87:3972-6, 1990.
Johnson et al., J. Virol., 67:438-445,1993.
Johnson et al., Mol. Cell. Biol., 9:3393, 1989.
Joyce, Nature, 338:217-244, 1989.
Ju et al., Gene Ther., 7(4):329-38, 2000.
Kadesch and Berg, Mol. Cell. Biol., 6:2593, 1986.
Kaeppler et al., Plant Cell Reports 9: 415-418, 1990.
Kamb et al., Nature Genetics, 8:22-26, 1994.
Kamb et al., Science, 2674:436-440, 1994.
Kamitani et al., J. Biol Chem., 272:28557-62, 1997.
Kaneda et al., Science, 243:375-378, 1989.
Karin et al., Mol. Cell. Biol., 7:606, 1987.
Katinka et al., Cell, 20:393, 1980.
Katinka et al., Nature, 290:720, 1981.
Kato et al, J. Biol. Chem., 266:3361-3364, 1991.
Kawamoto et al., Mol. Cell. Biol., 8:267, 1988.
Keane et al., Cancer Res. 59:734-41, 1999.
Keler et al., J Acquir Immune Defic Syndr Hum Retrovirol, 13:117-26, 1996.
Kerr et al., Br. J. Cancer, 26(4):239-57, 1972.
Kiledjian et al., Mol. Cell. Biol., 8:145, 1988.
Kim and Cech, Proc. Nat'l Acad. Sci. USA, 84:8788-8792, 1987.
Klamut et al., Mol. Cell. Biol., 10:193, 1990.
Koch et al., Mol. Cell. Biol., 9:303, 1989.
Kraus et al. FEBS Lett., 428(3):165-170, 1998.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kriegler et al., In: Gene Expression, D. Harner and M. Rosenberg, eds., New York: Alan R. Liss, 1983.
Kuhl et al., Cell, 50:1057, 1987.
Kura, Natl Med J India, 11:201-202, 1998.
Kunz et al., Nucl. Acids Res., 17:1121, 1989.
Kwoh et al., Proc. Nat. Acad. Sci. USA, 86: 1173, 1989.
Kyte and Doolittle, J. Mol. Biol., 157(1):105-132, 1982.
Landis et al., CA Cancer J. Clin., 48:6, 1998.
Laperche et al., Aids, 12:1397-8, 1998.
Lareyre et al., J. Biol Chem., 274(12):8282-8290, 1999.
Larsen et al., Proc. Nat'l Acad. Sci. USA., 83:8283, 1986.
Laspia et al., Cell, 59:283, 1989.
Latimer et al., Mol. Cell. Biol., 10:760, 1990.
Lee et al., J Auton Nerv Syst. 74(2-3):86-90, 1997.
Lee et al., Nature, 294:228, 1981.
Lee et al., Nucleic Acids Res., 12:4191-206, 1984.
Levenson et al., Hum Gene Ther. 20;9(8):1233-1236, 1998.
Levinson et al., Nature, 295:79, 1982.
Lieber and Strauss, Mol. Cell. Biol., 15: 540-551, 1995.
Lin et al., Mol. Cell. Biol., 10:850, 1990.
Litvak et al., Med Decis Making, 17:455-63, 1997.
Luque et al., Eur J Clin Microbiol Infect Dis, 12:663-7, 1993.
Luria et al., EMBO J., 6:3307, 1987.
Lusky and Botchan, Proc. Nat'l Acad. Sci. USA., 83:3609, 1986.
Lusky et al., Mol. Cell. Biol., 3:1108, 1983.
Macejak and Sarnow, Nature, 353:90-94, 1991.
Majors and Varmus, Proc. Nat'l Acad. Sci. USA., 80:5866, 1983.
Mannick et al., Science 284(5414), 651-4, 1999.
Marsters et al., Recent Prog Horm Res 54:225-34, 1999.
Martin et al., Nature, 345(6277):739-743, 1990.
Mayer et al., Biochimica et Biophlysica Acta, vol. 858, pp. 161-168, 1986.
Mayhew et al., Biochimica et Biophysica Acta, vol. 775, pp. 169-174, 1984.
Mayhew et al., Methods in Enzymology, vol. 149, pp. 64-77, 1987.
McNeall et al., Gene, 76:81, 1989.
Michel and Westhof, J. Mol. Biol., 216:585-610, 1990.
Miksicek et al., Cell, 46:203, 1986.
Mitchell et al., Ann. N.Y. Acad. Sci., 690:153-166, 1993.
Mitchell et al., J. Clin. Oncol,. 8(5):856-859, 1990.
Monzo et al., J Clin Oncol., 17:2100-4, 1999.
Mordacq and Linzer, Genes and Dev., 3:760, 1989.
Moreau et al., Nucl. Acids Res., 9:6047, 1981.
Mori et al., Cancer Res., 54:3396-3397, 1994.
Morton and Ravindranath, M. H. Current concepts concerning melanoma vaccines. In Tumor Immunology, Dalgleish A G (ed.), London: Cambridge University Press, 1-55, 1996.
Morton et al., Ann. Surg. 216: 463-482, 1992.
Moulding et al., Blood 92(7), 2495-502, 1998.
Muesing et al., Cell, 48:691, 1987.
Nakamura et al., In: Enzyme Immunoassays: Heterogeneous and Homogeneous Systems, Chapter 27, 1987.
Ng et al., Nuc. Acids Res., 17:601, 1989.
Nicolas and Rubenstein, In: Vectors: A survey of molecular cloning vectors and their uses, Rodriguez and Denhardt (eds.), Stoneham: Butterworth, pp. 493-513, 1988.
Nicolau and Sene, Biochim. Biophys. Acta, 721:185-190, 1982.
Nicolau et al., Methods Enzymol., 149:157-176, 1987.
Nigg, Nature, 386:779-87, 1997.
Nobri et al., Nature, 368:753-756, 1995.
Nomoto et al., Gene, 236(2):259-271, 1999.
Ohara et al., Proc. Nat'l Acad. Sci. USA, 86: 5673-5677, 1989.
Okamoto et al., Proc. Nat'l Acad. Sci. USA, 91:11045-11049, 1994.
Omirulleh et al., Plant Mol. Biol., 21:415-28, 1993.
Ondek et al., EMBO J., 6:1017, 1987.
Orlow et al., Cancer Res., 54:2848-2851, 1994.
Ornitz et al., Mol. Cell. Biol., 7:3466, 1987.
Palmiter et al., Nature, 300:611, 1982.
Palukaitis et al., Virology, 99:145-151, 1979.
Pan et al., J Infect Dis, 1991; 164:962-4,1991.
Patijn, Transpl Int, 6:165-72, 1993.
Pease et al., Proc. Natl. Acad. Sci. USA, 91:5022-5026, 1994.
Pech et al., Mol. Cell. Biol., 9:396, 1989.
Pelletier and Sonenberg, Nature, 334:320-325, 1988.
Pepose et al., Ophthalmology, 99:879-88, 1992.
Perez-Stable and Constantini, Mol. Cell. Biol., 10:1116, 1990.
Perriman. et al., Gene, 113:157-163, 1992.
Perrotta and Been, Biochemistry 31:16, 1992.
Pfeffer et al., Cell, 73:457-467, 1993.
Picard and Schaffner, Nature, 307:83, 1984.
Pietras et al., Oncogene, 17(17):2235-49, 1998.
Pinkert et al., Genes and Dev., 1:268, 1987.
Ponta et al., Proc. Nat'l Acad. Sci. USA., 82:1020, 1985.
Porton et al., Mol. Cell. Biol., 10:1076, 1990.
Potrykus et al., Mol. Gen. Genet., 199:183-188, 1985.
Prody, G. A. et al., Science, 231, 1577-1580, 1986.
Qin et al., Proc. Nat'l Acad. Sci. USA, 95(24):1411-6, 1998.
Queen and Baltimore, Cell, 35:741, 1983.
Quinn et al., Mol. Cell. Biol., 9:4713, 1989.
Race et al., Virology, 184:716-22, 1991.
Ravindranath and Morton, Intern. Rev. Immunol. 7: 303-329, 1991.
Redondo et al., Science, 247:1225, 1990.
Reinhold-Hurek and Shub, Nature, 357:173-176, 1992.
Reisman and Rotter, Mol. Cell Biol., 9:3571, 1989.
Remington's Phamaceutical Sciences, 15^{th} ed., pages 1035-1038 and 1570-1580, Mack Publishing Company, Easton, Pa., 1980.
Resendez Jr. et al., Mol. Cell. Biol., 8:4579, 1988.
Rich et al., J Acquir Immune Defic Syndr Hum Retrovirol, 17:376-9, 1998.
Ridgeway, In: Vectors: A survey of molecular cloning vectors and their uses, Rodriguez R L, Denhardt D T, ed., Stoneham:Butterworth, pp. 467-492, 1988.
Ripe et al., Mol. Cell. Biol., 9:2224, 1989.
Rippe et al., Mol. Cell Biol., 10:689-695, 1990.
Ritter et al., Am J Clin Pathol, 113:128-34, 2000.
Rittling et al., Nucl. Acids Res., 17:1619, 1989.
Rosen et al., Cell, 41:813, 1988.
Rosenberg et al., Ann. Surg., 210:474, 1989.
Rosenberg et al., N. Engl. J. Med., 319:1676, 1988.
Sakai et al., Genes and Dev., 2:1144, 1988.
Sambrook et al., In: Molecular Cloning: A Laboratory Manual, 2nd Ed., Cold Spring Harbor Press, Cold Spring Harbor, N.Y., 1989.
Sanford et al., Journal of National Cancer Institute, 9:229-246, 1948.
Sarver, et al, Science, 247:1222-1225, 1990.
Satake et al., J. Virology, 62:970, 1988.
Sato & Yamashina, Biochim Biophys Acta, 397:179-87, 1975.
Scanlon et al., Proc. Nat 'l Acad. Sci. USA, 88:10591-10595, 1991.
Schaffner et al., J. Mol. Biol., 201:81, 1988.
Schleupner, Principals and Practice of Infectious Diseases, 10:3-19, 1989.
Schmitt et al., Exp Cell Res., 240:107-21, 1998.
Searle et al., Mol. Cell. Biol., 5:1480, 1985.
Sehgal, Bull World Health Organ, 76:509-13, 1998.
Serrano et al., Nature, 366:704-707, 1993.
Serrano et al., Science, 267:249-252, 1995.
Sharp and Marciniak, Cell, 59:229, 1989.
Shaul and Ben-Levy, EMBO J., 6:1913, 1987.
Sheppard et al., J Acquir Immune Defic Syndr, 6:1339-46, 1993.
Sherman et al., Mol. Cell. Biol., 9:50, 1989.
Shinoda, K. et al., Colloidal Surfactant, Academic Press, especially “The Formation of Micelles”, Ch. 1, 1-96, 1963.
Shoemaker et al., Nature Genetics, 14:450-456, 1996.
Simmonds et al., J Virol, 64:864-72, 1990.
Simonds et al., N Engl J Med, 326:726-32, 1992.
Sioud et al., J. Mol. Biol., 223:831-835, 1992.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Steckelberg et al., Mayo Clin Proc, 63:373-80, 1988.
Stephens and Hentschel, Biochem. J., 248:1, 1987.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Symons, Ann. Rev. Biochem., 61:641-671, 1992.
Symons, Nucl. Acids Res., 9:6527-6537, 1981.
Szoka et al., Proc. Natl. Acad. Sci., 1978, 75:4194-4198.
Takebe et al., Mol. Cell. Biol., 8:466, 1988.
Taules et al., J Biol Chem., 274:24445-8, 1999.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (ed.), New York: Plenum Press, pp. 149-188, 1986.
Templeton et al., Nat. Biotechnol., 15(7):647-52, 1997.
Thiele et al., Eur. J. Biochem., 267:5473-81, 2000.
Thiesen et al., J. Virology, 62:614, 1988.
Thompson et al. Nature Medicine, 1:277-278, 1995.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsujimoto and Croce, Proc. Natl. Acad. Sci. USA, 83(14):5214-8, 1986.
Tsujimoto et al., Science, 228(4706):1440-3, 1985.
Tsumaki et al., J. Biol Chem. 273(36):22861-22864, 1998.
Tyndall et al., Nuc. Acids. Res., 9:6231, 1981.
Urnovitz, Nat Med, 3:1258, 1997.
Vannice and Levinson, J. Virology, 62:1305, 1988.
Vasseur et al., Proc. Nat'l Acad. Sci. USA., 77:1068, 1980.
von Heijne, Eur J Biochem 116:419-22, 1981.
Wada et al., Nucleic Acids Res., 18:2367-2411, 1990.
Walker et al., Proc. Nat'l Acad. Sci. USA, 89:392-396 1992.
Wang and Calame, Cell, 47:241, 1986.
Wawrzynczak & Thorpe, Cancer Treat. Res., 37:239-51, 1988.
Weber et al., Cell, 36:983, 1984.
Weinberger et al. Mol. Cell. Biol., 8:988, 1984.
Weinberger et al., Science, 228:740-742, 1985.
Winoto and Baltimore, Cell, 59:649, 1989.
Wolf et al., Comput. Appl. Biosci., 4(1):187-191, 1988.
Wong et al., Gene, 10:87-94, 1980.
Wu et al., Biochiem. Biophys. Res. Commun., 233(1):221-6, 1997.
Yerly, Schweiz Med Wochenschr, 129:319-22, 1999.
Yuan and Altman, Science, 263:1269-1273, 1994.
Yuan et al., Proc. Nat 'l Acad. Sci. USA, 89:8006-8010, 1992.
Yutzey et al. Mol. Cell. Biol., 9:1397, 1989.
Zazzi et al., AIDS Res Hum Retroviruses, 9:315-20, 1993.
Zazzi et al., J Clin Microbiol, 33:205-8, 1995.
Zhao-Emonet et al., Biochem. Biophys. Acta., 1442(2-3):109-19, 1998.
Zoldhelyi, Circulation 93, 10-17, 1996.
Zoldhelyi et al., Circulation 101(3), 289-95, 2000.
Zhou et al., Blood, 89:630-43, 1997.

1. A method of screening for human immunodeficiency virus in a subject comprising:
a) contacting a sample from the subject with a composition comprising a recombinant, nondenatured human immunodeficiency virus gp41 antigen under conditions that permit formation of an immunocomplex between any antibody in the sample that can specifically bind to the gp41 antigen; and
b) detecting whether an immunocomplex is formed between an antibody and the gp41 antigen.

2. The method of claim 1, wherein the antigen is prepared by a process of solubilizing the gp41 antigen with a composition comprising digitonin, NP40, or deoxycholate.

3. The method of claim 2, wherein the composition comprises digitonin.

4. The method of claim 3, wherein the concentration of digitonin in the composition is 0.1% to 10.0%.

5. The method of claim 4, wherein the concentration of digitonin in the composition is about 1.0%.

6. The method of claim 2, wherein the composition comprises NP40.

7. The method of claim 6, wherein the concentration of NP40 in the composition is 0.2% to 5.0%.

8. The method of claim 7, wherein the concentration of NP40 in the composition is about 2.0%.

9. The method of claim 2, wherein the composition comprises deoxycholate.

10. The method of claim 9, wherein the concentration of deoxycholate in the composition is 0.05% to 0.5%.

11. The method of claim 10, wherein the concentration of deoxycholate is about 0.1%.

12. The method of claim 1, wherein the recombinant, nondenatured human immunodeficiency virus gp41 antigen is substantially purified.

13. The method of claim 1, wherein the gp41 antigen comprises 10 contiguous amino acids of amino acid sequence 511 to 856 of SEQ ID NO:1.

14. The method of claim 13, wherein the gp41 antigen comprises 30 contiguous amino acids of amino acid sequence 511 to 856 of SEQ ID NO:1.

15. The method of claim 14, wherein the gp41 antigen comprises 50 contiguous amino acids of amino acid sequence 511 to 856 of SEQ ID NO:1.

16. The method of claim 15, wherein the gp41 antigen comprises amino acid sequence 511 to 856 of SEQ ID NO:1.

17. The method of claim 1, wherein the gp41 antigen comprises at least 5 contiguous amino acids of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

18. The method of claim 1, wherein the composition further comprises a recombinant, nondenatured human immunodeficiency virus gp160 antigen under conditions that permit formation of an immunocomplex between any antibody in the sample that can bind to the gp160 antigen.

19. The method of claim 18, further comprising
c) detecting whether an immunocomplex between an antibody in the sample and the gp160 antigen is formed.

20. The method of claim 19, wherein the gp160 antigen comprises 20 contiguous amino acid of SEQ ID NO:1.

21. The method of claim 20, wherein the gp160 antigen comprises 50 contiguous amino acid of SEQ ID NO:1.

22. The method of claim 21, wherein the gp160 antigen comprises 100 contiguous amino acid of SEQ ID NO:1.

23. The method of claim 22, wherein the gp160 antigen comprises SEQ ID NO:1.

24. The method of claim 18, wherein the gp41 or gp160 antigen is from HIV₂₁₃, HIV_{AC-1}, HIV_{C}, or a combination thereof.

25. The method of claim 19, wherein the recombinant, nondenatured gp41 or gp160 antigen is capable of specifically binding an antibody from the sample 2 to 50 days earlier than a denatured gp41 or gp160 antigen.

26. The method of claim 19, wherein the recombinant, nondenatured gp41 or gp160 antigen is capable of specifically binding an antibody from a sample that is obtained 2 to 50 days earlier than a second sample in which a denatured gp41 or gp160 antigen is capable of specifically binding an antibody in the second sample.

27. The method of claim 26, wherein the recombinant, nondenatured gp41 or gp160 antigen is capable of specifically binding an antibody from the sample that is obtained at least 15 days earlier than a second sample in which a denatured gp41 or gp160 antigen is capable of specifically binding an antibody in the second sample.

28. The method of claim 27, wherein the recombinant, nondenatured gp41 or gp160 antigen is capable of specifically binding an antibody from the sample that is obtained at least 30 days earlier than a second sample in which a denatured gp41 or gp 160 antigen is capable of specifically binding an antibody in the second sample.

29. The method of claim 1, wherein the sample was obtained fewer than 16 weeks subsequent to HIV infection.

30. The method of claim 1, wherein a denatured gp41 antigen is not capable of forming an immunocomplex with an antibody in the sample.

31. The method of claim 19, wherein a denatured gp160 antigen is not capable of forming an ininunocomplex with an antibody in the sample.

32. The method of claim 1, wherein the immunocomplex is detected using anti-antibody secondary reagents.

33. The method of claim 1, wherein the immunocomplex is detected by ELISA.

34. The method of claim 1, wherein the immunocomplex is detected by Western blotting.

35. The method of claim 1, wherein the subject is a human.

36. The method of claim 35, wherein the subject is an infant.

37. The method of claim 36, wherein the antibody is an IgA antibody.

38. The method of claim 36, wherein the antibody is an IgM antibody.

39. The method of claim 1, wherein the recombinant, nondenatured human immunodeficiency virus gp41 antigen is obtained from a mammalian cell comprising a recombinant vector comprising a nucleic acid sequence encoding the gp41 antigen.

40. The method of claim 39, wherein the mammalian cell is a CEM or Mu-1-Lu cell.

41. The method of claim 1, wherein the wherein the recombinant, nondenatured human immunodeficiency virus gp41 antigen is obtained from an insect cell comprising a baculovirus vector comprising a nucleic acid sequence encoding the gp41 antigen.

42. The method of claim 18, wherein the recombinant, nondenatured human immunodeficiency virus gp160 antigen is obtained from a mammalian or insect cell comprising a recombinant vector comprising a nucleic acid sequence encoding the gp160 antigen.

43. The method of claim 1, wherein the subject is afflicted with idiopathic chronic lymphopenia.

44. A kit for screening for early human immunodeficiency virus antibodies, in a suitable container means, comprising:
a) a recombinant, nondenatured gp41 antigen; and
b) a recombinant, nondenatured gp160 antigen.

45. The kit of claim 44, further comprising a nonreactive solid support to which the gp41 and gp160 antigens are attached.

46. The kit of claim 45, further comprising a first agent that detects an immunocomplex comprising the gp41 antigen and a second agent that detects an immunocomplex comprising the gp160 antigen.

47. The kit of claim 46, wherein the first and second agents are secondary antibodies that specifically bind early human immunodeficiency virus antibodies.

48. The kit of claim 46, wherein the first and second agents comprise a detectable label.

49. The kit of claim 48, wherein the detectable label is fluorescent, radioactive, colorimetric, or enzymatic.

50. The kit of claim 44, wherein the gp41 antigen comprises 20 contiguous amino acids of amino acid sequence 511 to 856 of SEQ D NO:1.

51. The kit of claim 50, wherein the gp41 antigen comprises 30 contiguous amino acids of amino acid sequence 511 to 856 of SEQ ID NO:1.

52. The kit of claim 51, wherein the gp41 antigen comprises 50 contiguous amino acids of amino acid sequence 511 to 856 of SEQ ID NO:1.

53. The kit of claim 52, wherein the gp41 antigen comprises amino acid sequence 511 to 856 of SEQ ID NO:1.

54. The kit of claim 52, wherein the gp41 antigen comprises 5 contiguous amino acids of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

55. The kit of claim 54, wherein the gp41 antigen comprises the amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, or SEQ ID NO:12.

56. The kit of claim 44, wherein the gp160 antigen comprises 20 contiguous amino acid of SEQ ID NO:1.

57. The kit of claim 56, wherein the gp160 antigen comprises 50 contiguous amino acid of SEQ ID NO:1.

58. The kit of claim 57, wherein the gp160 antigen comprises 100 contiguous amino acid of SEQ ID NO:1.

59. A kit for screening for early anti-human immunodeficiency virus antibodies in a subject comprising:
a) an assay play comprising a multiplicity of microtiter wells comprising a composition comprising a recombinant, nondenatured human immunodeficiency virus gp41 antigen capable of binding an human deficiency virus early antibody in the sample that can specifically bind to gp41; and
b) a container means comprising a labeled secondary antibody having specific binding affinity for a human irnmunodeficiency early antibody in the sample that can specifically bind to gp41.

60. The kit of claim 59, wherein the composition further comprises a recombinant, nondenatured human immunodeficiency virus gp160 antigen capable of specifically binding an early human immunodeficiency virus antibody that can specifically bind gp160.

61. The kit of claim 60, further comprising a labeled secondary antibody having specific binding affinity for a human immunodeficiency early antibody in the sample that can specifically bind to gp160.

62. A method of screening for human immunodeficiency virus in a subject comprising:
a) contacting a sample from the subject with a composition from the kit of claim 44; and,
b) detecting whether an imnuunocomplex is formed between an antibody and the gp41 antigen or the gp160 antigen.


SEQUENCE LISTING (12 sequences)

SEQ ID NO: 1 - protein, 856 aa, HIV-1
MRVKEKYQHLRRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGGMIMEKGEIKNCSFNISTSIRGKVQKEYAFFYKHDIIPIDNDTTSYTLTSCNTSVITQACPKVSFEPIPIHYCAPAGFAILKCNNKTFNGTGPCTNVSTVQCTHGIKPVVSTQLLLNGSLAEEEVVIRSANLTDNVKTIIVQLNQSVEINCTRPNNNTRKRIRIQRGPGRTFVTIGKIGNMRQAHCNISRAKWNNTLKQIASKLREQYGNNKTIIFKQSSGGDLEIVTHSFNCGGEFFYCNSTQLFNSTWFNSTWSTEGSNNTEGSDTITLPCRIKQIINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKIFIMIVGGLVGLRIVFAVLSIVNRVRQGHSPLSFQTHLPTPGGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 346 aa, HIV-1
RAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKIFIMIVGGLVGLRIVFAVLSIVNRVRQGHSPLSFQTHLPTPGGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 3 - protein, 19 aa, HIV-1
KDQQLLGIWGCSGKLICTT

SEQ ID NO: 4 - protein, 18 aa, HIV-1
KDQQLLGIWGCKGKICYT

SEQ ID NO: 5 - protein, 23 aa, HIV-1
NQQLLSLWGCKGKLVCYTSVKWN

SEQ ID NO: 6 - protein, 23 aa, HIV-1
NQQLLGIWGCSGKLICTTAVPWN

SEQ ID NO: 7 - protein, 36 aa, HIV-1
ARILAVERYLKDQQLLGIWGCSGKLICTTAVPWNAS

SEQ ID NO: 8 - protein, 11 aa, HIV-1
GIWGCSGKLIC

SEQ ID NO: 9 - protein, 21 aa, HIV-1
RILAVERYLKDQQLLGIWGCS

SEQ ID NO: 10 - protein, 12 aa, HIV-1
LGLWGCSGKLIC

SEQ ID NO: 11 - protein, 15 aa, HIV-1
SGKLICTTAVPWNAS

SEQ ID NO: 12 - protein, 35 aa, HIV-1
RILAVERYLKDQQLLGIWGCSGKLICTTAVPWNAS